SEQUENCE LISTING (8 sequences)

SEQ ID NO: 1 - DNA, 1260 bp, synthetic construct
  misc_feature 1..1260: note = Synthetic DNA
  source 1..1260: mol_type = other DNA
  CDS 1..1260
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 2 - protein, 419 aa, synthetic construct
  REGION 1..419: note = Synthetic Construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - RNA, 1260 nt, synthetic construct
  misc_feature 1..1260: note = synthetic RNA
  source 1..1260: mol_type = other RNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 4 - DNA, 3822 bp, synthetic construct
  misc_feature 1..3822: note = synthetic DNA
  source 1..3822: mol_type = other DNA
  CDS 1..3822
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctccagaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 5 - protein, 1273 aa, synthetic construct
  REGION 1..1273: note = Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - RNA, 3822 nt, synthetic construct
  misc_feature 1..3822: note = synthetic RNA
  source 1..3822: mol_type = other RNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctccagaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 7 - DNA, 22 bp, synthetic construct
  misc_feature 1..22: note = synthetic oligonucleotide
  source 1..22: mol_type = other DNA
ggaagagacaggtacgttaata

SEQ ID NO: 8 - DNA, 21 bp, synthetic construct
  misc_feature 1..21: note = synthetic oligonucleotide
  source 1..21: mol_type = other DNA
agcagtacgcacacaatcgaa

(19) United States
(12) Patent Application Publication
(10) Pub. No.: US 2026/0041759 A1
(43) Pub. Date: Feb. 12, 2026

(54) COVID19 mRNA Vaccine

(71) Applicants: Board of Regents, The University of Texas System, Austin, TX (US); University of Pennsylvania, Philadelphia, PA (US)

(72) Inventors: Haitao Hu, San Antonio, TX (US); Drew Weissman, Philadelphia, PA (US)

(21) Appl. No.: 18/696,353

(22) PCT Filed: Sep. 30, 2022
(86) PCT No.: PCT/US2022/045424

§ 371 Date: Mar. 27, 2024

Related U.S. Application Data
(60) Provisional application No. 63/251,384, filed on Oct. 1, 2021.
(60) Provisional application No. 63/331,925, filed on Apr. 18, 2022.

(51) Int. Cl. A61K 39/215; A61K 9/1272; A61K 9/51; A61K 39/00; A61P 31/14

(52) U.S. Cl. CPC A61K 39/215; A61K 9/1272; A61K 9/5123; A61P 31/14; A61K 2039/53; A61K 2039/545; A61K 2039/55555; C12N 2770/20034

(57) ABSTRACT

A solution has been discovered that provides a more effective Coronavirus vaccine. The solution is an mRNA vaccine encoding a SARS-CoV-2 nucleoprotein (N) (mRNA-N) in combination with an mRNA vaccine encoding SARS-CoV-2 spike protein(S) (mRNA-S). Chemically modified mRNA-N (pseudouridine) and/or chemically modified mRNA-S (pseudouridine) can be synthesized and packaged in lipid nanoparticles (LNP). In mouse and hamster models, it was shown that mRNA-N alone is immunogenic and can significantly diminish viral loads in the mouse lung after prime-boost intramuscular immunization. In addition, the combinatorial mRNA-N/mRNA-S vaccination induces substantially stronger protection against SARS-CoV-2 than vaccination with mRNA-S alone.

### PRIORITY PARAGRAPH

This application is a 371 filing of international application PCT/US2022/045424 filed Sep. 30, 2022 claiming priority to U.S. Provisional Applications 63/251,384 filed Oct. 1, 2021 and 63/331,925 filed Apr. 18, 2022, each of which are incorporated herein by reference in their entirety.

### STATEMENT REGARDING FEDERALLY FUNDED RESEARCH

None.

### REFERENCE TO SEQUENCE LISTING

A sequence listing required by 37 CFR 1.821-1.825 is being submitted electronically with this application. The sequence listing is incorporated herein by reference. The sequence listing that is contained in the file named “UTMBP0407US” which is 23 KB (as measured in Microsoft Windows®) and was created on Nov. 13, 2024.

### BACKGROUND

SARS-CoV-2 is a coronavirus that was first discovered late 2019 in the Wuhan region in China. SARS-CoV-2 is a beta-coronavirus, like MERS-CoV and SARS-CoV, all of which have their origin in bats. There are currently several sequences available from several patients from the U.S., China and other countries, suggesting a likely single, recent emergence of this virus from an animal reservoir. The name of this disease caused by the virus is coronavirus disease 2019, abbreviated as COVID-19. Symptoms of COVID-19 range from mild symptoms to severe illness and death for confirmed COVID-19 cases.

As indicated above, SARS-CoV-2 has strong genetic similarity to bat coronaviruses, from which it likely originated, although an intermediate reservoir host such as a pangolin is thought to be involved. From a taxonomic perspective SARS-CoV-2 is classified as a strain of the severe acute respiratory syndrome (SARS)-related coronavirus species.

Coronaviruses are enveloped RNA viruses. The major surface protein is the large, trimeric spike glycoprotein(S) that mediates binding to host cell receptors as well as fusion of viral and host cell membranes. The S protein is composed of an N-terminal S1 subunit and a C-terminal S2 subunit, responsible for receptor binding and membrane fusion, respectively. Recent cryo-EM reconstructions of the CoV trimeric S structures of alpha-, beta-, and deltacoronaviruses revealed that the 51 subunit comprises two distinct domains: an N-terminal domain (51 NTD) and a receptor-binding domain (51 RBD). SARS-CoV-2 makes use of its 51 RBD to bind to human angiotensin-converting enzyme 2 (ACE2).

The rapid expansion of the COVID-19 pandemic has made the development of a SARS-CoV-2 vaccine a global health priority. Since the novel SARS-CoV-2 virus was first observed in humans in late 2019, over 8 million people have been infected and hundreds of thousands have died as a result of COVID-19. The World Health Organization declared the 2019-nCoV outbreak a Public Health Emergency of International Concern on Jan. 30, 2020.

Current COVID19 vaccines, including the FDA-approved Pfizer/BioNtech mRNA vaccine, largely target the viral spike protein(S). With the emergence and fast spread of viral variants, vaccine approaches targeting only S protein appear to become less effective.

There remains a need for additional Coronavirus vaccines and methods for administering these vaccines particularly since variants of the SARS-CoV-2 are being identified.

### SUMMARY

Additional Coronavirus vaccines and vaccine combinations are described herein. Certain embodiments are directed to an mRNA vaccine encoding a SARS-CoV-2 nucleoprotein (N) (mRNA-N). Chemically modified mRNA-N (pseudouridine) was synthesized and packaged in lipid nanoparticles (LNP). In a mouse model, it was shown that mRNA-N is immunogenic and can significantly diminish viral loads in the mouse lung after prime-boost intramuscular immunization. mRNA-N CoVID19 vaccine approach can be complementary to the approved COVID19 vaccines for combating SARS-CoV-2 and its variants.

Certain embodiments are directed to a SARS-CoV-2 vaccine, comprising an engineered messenger ribonucleic acid (mRNA) comprising an open reading frame encoding a coronavirus nucleoprotein (N) protein (mRNA-N). The N protein can have an amino acid sequence that is 90, 91, 92, 93, 94, 95, 96, 97, 98, 99, or 100% identical to the amino acid sequence of SEQ ID NO:2. The N protein can be encoded by a DNA is 80, 85, 90, 95, 96, 97, 98, 99, to 100% identical to atgtctgataatggaccccaaaatcagcgaaatgcaccccgcattacgtttggtggaccctcagattcaactggcagtaaccagaatggaga acgcagtggggcgcgatcaaaacaacgtcggccccaaggtttacccaataatactgcgtcttggttcaccgctctcactcaacatggcaag gaagaccttaaattccctcgaggacaaggcgttccaattaacaccaatagcagtccagatgaccaaattggctactaccgaagagctacca gacgaattcgtggtggtgacggtaaaatgaaagatctcagtccaagatggtatttctactacctaggaactgggccagaagctggacttccct atggtgctaacaaagacggcatcatatgggttgcaactgagggagccttgaatacaccaaaagatcacattggcacccgcaatcctgctaa caatgctgcaatcgtgctacaacttcctcaaggaacaacattgccaaaaggcttctacgcagaagggagcagaggcggcagtcaagcctct tctcgttcctcatcacgtagtcgcaacagttcaagaaattcaactccaggcagcagtaggggaacttctcctgctagaatggctggcaatggc ggtgatgctgctcttgctttgctgctgcttgacagattgaaccagcttgagagcaaaatgtctggtaaaggccaacaacaacaaggccaaact gtcactaagaaatctgctgctgaggcttctaagaagcctcggcaaaaacgtactgccactaaagcatacaatgtaacacaagctttcggcag acgtggtccagaacaaacccaaggaaattttggggaccaggaactaatcagacaaggaactgattacaaacattggccgcaaattgcaca atttgcccccagcgcttcagcgttcttcggaatgtcgcgcattggcatggaagtcacaccttcgggaacgtggttgacctacacaggtgccat caaattggatgacaaagatccaaatttcaaagatcaagtcattttgctgaataagcatattgacgcatacaaaacattcccaccaacagagcct aaaaaggacaaaaagaagaaggctgatgaaactcaagccttaccgcagagacagaagaaacagcaaactgtgactcttcttcctgctgca gatttggatgatttctccaaacaattgcaacaatccatgagcagtgctgactcaactcaggcctaa (SEQ ID NO:1) or its RNA counterpart (SEQ ID NO:3).

In another embodiment a vaccine can comprises an engineered messenger ribonucleic acid (mRNA) having an open reading frame encoding a coronavirus spike(S) protein (mRNA-S). The S protein can have an amino acid sequence that is 90, 91, 92, 93, 94, 95, 96, 97, 98, 99, or 100% identical to the amino acid sequence of SEQ ID NO:5. The S protein can be encoded by a DNA is to 80, 85, 90, 95, 96, 97, 98, 99, to 100% identical atgtttgtttttcttgttttattgccactagtctctagtcagtgtgttaatcttacaaccagaactcaattaccccctgcatacactaattctttcacacg tggtgtttattaccctgacaaagttttcagatcctcagttttacattcaactcaggacttgttcttacctttcttttccaatgttacttggttccatgctat acatgtctctgggaccaatggtactaagaggtttgataaccctgtcctaccatttaatgatggtgtttattttgcttccactgagaagtctaacata ataagaggctggatttttggtactactttagattcgaagacccagtccctacttattgttaataacgctactaatgttgttattaaagtctgtgaattt caattttgtaatgatccatttttgggtgtttattaccacaaaaacaacaaaagttggatggaaagtgagttcagagtttattctagtgcgaataattg cacttttgaatatgtctctcagccttttcttatggaccttgaaggaaaacagggtaatttcaaaaatcttagggaatttgtgtttaagaatattgatg gttattttaaaatatattctaagcacacgcctattaatttagtgcgtgatctccctcagggtttttcggctttagaaccattggtagatttgccaatag gtattaacatcactaggtttcaaactttacttgctttacatagaagttatttgactcctggtgattcttcttcaggttggacagctggtgctgcagctt attatgtgggttatcttcaacctaggacttttctattaaaatataatgaaaatggaaccattacagatgctgtagactgtgcacttgaccctctctca gaaacaaagtgtacgttgaaatccttcactgtagaaaaaggaatctatcaaacttctaactttagagtccaaccaacagaatctattgttagattt cctaatattacaaacttgtgcccttttggtgaagtttttaacgccaccagatttgcatctgtttatgcttggaacaggaagagaatcagcaactgt gttgctgattattctgtcctatataattccgcatcattttccacttttaagtgttatggagtgtctcctactaaattaaatgatctctgctttactaatgtc tatgcagattcatttgtaattagaggtgatgaagtcagacaaatcgctccagggcaaactggaaagattgctgattataattataaattaccaga tgattttacaggctgcgttatagcttggaattctaacaatcttgattctaaggttggtggtaattataattacctgtatagattgtttaggaagtctaat ctcaaaccttttgagagagatatttcaactgaaatctatcaggccggtagcacaccttgtaatggtgttgaaggttttaattgttactttcctttaca atcatatggtttccaacccactaatggtgttggttaccaaccatacagagtagtagtactttcttttgaacttctacatgcaccagcaactgtttgtg gacctaaaaagtctactaatttggttaaaaacaaatgtgtcaatttcaacttcaatggtttaacaggcacaggtgttcttactgagtctaacaaaa agtttctgcctttccaacaatttggcagagacattgctgacactactgatgctgtccgtgatccacagacacttgagattcttgacattacaccat gttcttttggtggtgtcagtgttataacaccaggaacaaatacttctaaccaggttgctgttctttatcaggatgttaactgcacagaagtccctgt tgctattcatgcagatcaacttactcctacttggcgtgtttattctacaggttctaatgtttttcaaacacgtgcaggctgtttaataggggctgaac atgtcaacaactcatatgagtgtgacatacccattggtgcaggtatatgcgctagttatcagactcagactaattctcctcggcgggcacgtag tgtagctagtcaatccatcattgcctacactatgtcacttggtgcagaaaattcagttgcttactctaataactctattgccatacccacaaatttta ctattagtgttaccacagaaattctaccagtgtctatgaccaagacatcagtagattgtacaatgtacatttgtggtgattcaactgaatgcagca atcttttgttgcaatatggcagtttttgtacacaattaaaccgtgctttaactggaatagctgttgaacaagacaaaaacacccaagaagtttttgc acaagtcaaacaaatttacaaaacaccaccaattaaagattttggtggttttaatttttcacaaatattaccagatccatcaaaaccaagcaaga ggtcatttattgaagatctacttttcaacaaagtgacacttgcagatgctggcttcatcaaacaatatggtgattgccttggtgatattgctgctag agacctcatttgtgcacaaaagtttaacggccttactgttttgccacctttgctcacagatgaaatgattgctcaatacacttctgcactgttagcg ggtacaatcacttctggttggacctttggtgcaggtgctgcattacaaataccatttgctatgcaaatggcttataggtttaatggtattggagtta cacagaatgttctctatgagaaccaaaaattgattgccaaccaatttaatagtgctattggcaaaattcaagactcactttcttccacagcaagtg cacttggaaaacttcaagatgtggtcaaccaaaatgcacaagctttaaacacgcttgttaaacaacttagctccaattttggtgcaatttcaagt gttttaaatgatatcctttcacgtcttgaccctccagaggctgaagtgcaaattgataggttgatcacaggcagacttcaaagtttgcagacatat gtgactcaacaattaattagagctgcagaaatcagagcttctgctaatcttgctgctactaaaatgtcagagtgtgtacttggacaatcaaaaag agttgatttttgtggaaagggctatcatcttatgtccttccctcagtcagcacctcatggtgtagtcttcttgcatgtgacttatgtccctgcacaag aaaagaacttcacaactgctcctgccatttgtcatgatggaaaagcacactttcctcgtgaaggtgtctttgtttcaaatggcacacactggtttg taacacaaaggaatttttatgaaccacaaatcattactacagacaacacatttgtgtctggtaactgtgatgttgtaataggaattgtcaacaaca cagtttatgatcctttgcaacctgaattagactcattcaaggaggagttagataaatattttaagaatcatacatcaccagatgttgatttaggtga catctctggcattaatgcttcagttgtaaacattcaaaaagaaattgaccgcctcaatgaggttgccaagaatttaaatgaatctctcatcgatct ccaagaacttggaaagtatgagcagtatataaaatggccatggtacatttggctaggttttatagctggcttgattgccatagtaatggtgacaa ttatgctttgctgtatgaccagttgctgtagttgtctcaagggctgttgttcttgtggatcctgctgcaaatttgatgaagacgactctgagccagt gctcaaaggagtcaaattacattacacataa (SEQ ID NO:4) or its RNA counterpart (SEQ ID NO:6). In certain aspects an mRNA vaccine can encode a polyprotein comprising an N protein and an S protein. The polyprotein can comprise a self-cleaving or protease cleavage site between the N and S proteins.

In certain embodiments the mRNA-N and mRNA-S vaccines are administered separately. In certain aspects mRNA-N vaccine is comprised in a first lipid or carrier and the mRNA-S vaccine is comprised in a second lipid or carrier. The mRNA-N lipid or carrier and mRNA-S lipid or carrier composition can be formulated in the same (co-formulation) or different formulations. The mRNA-N and mRNA-S can be co-administered. The phrase “co-administered” refers to administration of two compositions (e.g., mRNA-N and mRNA-S) to a patient or subject within a certain desired time. In certain aspects the two compositions are administered concurrently. In other aspects the two compositions are administered with 1, 2, 3, 4, 5, 6, 7, 8, 9, 10, 11, 12, 13, 14, 15, 16, 17, 18, 19 or 20 minutes, hours, days, weeks, or months (including all values and ranges there between). The mRNA-N, mRNA-S, or mRNA-N and mRNA-S composition(s) can be administered 1, 2, 3, 4, 5, 6, 7, 8, 9, 10 or more times with 1, 2, 3, 4, 5, 6, 7, 8, 9, 10, 11, 12, 13, 14, 15, 16, 17, 18, 19 or 20 minutes, hours, days, weeks, or months between administrations.

In certain aspects the mRNA is a linear or circular RNA. The RNA or DNA can have one or more of a 5′ UTR, a 3′ UTR, and/or a polyadenylation segment or site.

Certain embodiments are directed to a DNA construct encoding an mRNA-N vaccine, an mRNA-S vaccine, or both an mRNA-N and mRNA-S as described herein.

Certain embodiments are directed to a lipid nanoparticle (LNP) comprising one or more mRNA vaccines described herein. The LNP can comprise one or more of an ionizable cationic lipid, phosphatidylcholine, cholesterol, PEG-lipid, or any combination thereof.

Certain aspects of the invention are directed to a nucleoside-modified, CoVID19 mRNA vaccine that encodes a more conserved protein of the SARS-CoV-2, viral nucleoprotein (named as mRNA-N). In addition, combining mRNA-N with the clinically approved mRNA-S for vaccination (mRNA-S+N) induces robust protection against SARS-CoV-2 Delta and Omicron variants. Thus, the described technology presents a next-generation vaccine approach for SARS-CoV-2 variants.

Other embodiments are directed to methods of inducing an antigen-specific immune response in a subject, the method comprising administering to the subject one or more mRNA vaccine described herein to produce an antigen-specific immune response in the subject. The mRNA vaccine(s) can be administered at a dose of 0.1, 0.5, 1, 5, 10 mg mRNA per dose, or any value or range there between. In particular aspects an mRNA is administered at a dose of 0.5 to 2 μg per dose. In certain aspects the vaccine(s) are administered using a prime-boost regimen. The boost dose can be administered 2, 3, 4, 5 or 6 weeks after the prime dose.

A “nucleic acid vaccine” refers to a vaccine that includes a heterologous nucleic acid molecule under the control of a promoter for expression in a subject. The heterologous nucleic acid molecule can be incorporated into an expression vector, such as a plasmid. A “DNA vaccine” refers to a vaccine in which the nucleic acid is DNA. An “RNA vaccine” refers to a vaccine in which the nucleic acid is RNA (e.g., an mRNA).

“Percent (%) sequence identity” or “sequence % identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Depicts serological analysis 3 weeks after prime dose in mice.

FIG. 2. Depicts mRNA-N induced protection in mice.

FIG. 3. Depicts mRNA-N induced protection in Hamsters (SARS-CoV-2 delta variant).

FIG. 4. Depiction of combinatorial mRNA vaccination study against SARS-CoV-2 delta variant.

FIG. 5. Depicts viral copies in lung quantified by PCR 2 days post infection (2 DPI).

FIG. 6. Depicts viral copies (Log 10) in lung quantified by PCR 4 days post infection (4 DPI).

FIG. 7. mRNA-N vaccine immunogenicity in mice. (a) Analysis of total CD4⁺ and CD8⁺ T cell activation in the mouse spleen following mock or mRNA-N vaccination. Splenocytes collected at week 5 (2 weeks after booster) were stained for mouse CD3, CD4, CD8, and CD44. Expression of CD44 on total CD4⁺ and CD8⁺ T cells were examined by flow cytometry and shown as % CD44⁺ in CD4⁺ or CD8⁺ T cells. (b) ICS measurement of vaccine-specific T cells in mouse spleen. Representative FACS plots for cytokine expression in T cells were shown. (c-d) Comparison of % cytokine-positive, N-specific CD4⁺ (c) or CD8⁺ (d) T cells in the spleen between mock and mRNA-N vaccine groups. (e) Comparison of levels of N-specific T cells in the spleen measured by IFN-γ ELISPOT. Data were shown as spot forming cells (SFC) per 10⁶ splenocytes; error bars showed SD of the mean. (f) ELISA measurement of serum N-specific binding IgG following prime (week 3) or booster (week 5) vaccination. OD values for individual serum samples after prime or booster vaccination at the indicated serum dilution (1:2700 for prime; 1:72900 for booster) were shown. (g) Comparison of N-specific binding IgG endpoint titers (EPT) between mock and vaccine groups after prime and booster vaccination. (h) Serum neutralizing activity measured by Plaque Reduction Neutralization Test (PRNT) using WT SARS-CoV-2. PRNT₅₀ for individual serum samples of the mock and vaccine groups were shown. LOD: limit of detection. Negative control and positive control were included. One-way ANOVA or Student's t-test were used for statistical analysis. * p<0.05, ** p<0.01, *** p<0.001, **** p<0.0001.

FIG. 8. mRNA-N induced protection in mice and hamsters. (a) SARS-CoV-2 viral RNA copies (Log₁₀) in the lung of mice after vaccination and challenge. Balb/c mice (8/group) were vaccinated with mock and mRNA-N at week 0 and week 3, followed by intranasal challenge with a mouse-adapted SARS-CoV-2 strain (2×10⁴ pfu). Two days post-infection (2 DPI), absolute viral RNA copies in the lung were quantified by qRT-PCR that included a standard curve and were compared between the mock and vaccine group. (b) SARS-CoV-2 viral RNA copies (Logo) in the lung of hamsters after vaccination and challenge. Hamsters (12/group) were vaccinated with mock and mRNA-N at week 0 and week 3, followed by intranasal challenge with the SARS-CoV-2 Delta strain (2×10⁴ pfu). On 2 DPI (n=6) and 4 DPI (n=6), absolute viral RNA copies in the lung were quantified and compared between the mock and vaccine group. (c) Comparison of weight loss for hamsters between mock and vaccine group from Day 0 to 4 DPI (mean±SD). (d) SARS-CoV-2 viral RNA copies (Logo) in the lung of hamsters after vaccination and challenge. Data for mock, mRNA-N, and mRNA-N with CD8 depletion groups were shown. One-way ANOVA or Student's t-test were used for statistical analysis. * p<0.05, ** p<0.01, *** p<0.001, **** p<0.0001.

FIG. 9. Protection induced by combination mRNA-S+N vaccination against Delta variant compared to mRNA-S alone. (a) Viral RNA copies (Logo) in the lung of mice after vaccination and challenge. Balb/c mice (8/group) were vaccinated with mock, mRNA-S alone, and combination mRNA-S+N at week 0 and week 3, followed by intranasal challenge with a mouse-adapted SARS-CoV-2 strain (2×10⁴ pfu). On 2 DPI, absolute viral RNA copies in the lung were quantified. (b-c) Viral RNA copies (Log₁₀) in the lung (b) or nasal wash (c) of hamsters after vaccination and challenge. Hamsters (12/group) were vaccinated with mock, mRNA-S alone, and combination mRNA-S+N at week 0 and week 3, followed by intranasal challenge with the Delta strain (2×10⁴ pfu). On 2 DPI (n=6) and 4 DPI (n=6), absolute viral RNA copies in the lung (b) or in nasal wash (d) were quantified. (d) Comparison of weight loss for hamsters between mock and vaccine groups from Day 0 to Day 4 after viral infection (DPI) (mean±SD). One-way ANOVA was used for statistical analysis. LOD: limit of detection (102 copies). * p<0.05, ** p<0.01, *** p<0.001, **** p<0.0001.

FIG. 10. Protection induced by combination mRNA-S+N vaccination against Omicron variant. Four groups of hamsters (n=10; Groups 1-4) were vaccinated with empty LNP (mock), mRNA-S (2 μg), mRNA-S (4 μg), or mRNA-S+N (2 μg for each) at week 0 (prime) and week 3 (boost) via the i.m. route (a-d). A fifth group (n=10) that was vaccinated with mRNA-S+N (2 μg for each) but received two doses of CD8 depletion antibody prior to viral challenge (−D6 and −D3) was also included to explore role of CD8 cells in protection (e-f). At 2 weeks after booster vaccination (week 5), all hamsters were intranasally challenged with SARS-CoV-2 Omicron strain (2×10⁴ pfu). (a-b) Viral RNA copies (Log₁₀) in the lung of hamsters (Groups 1-4) after viral challenge on 2 DPI (n=5) (a) and 4 DPI (n=5) (b). (c) Viral RNA copies (Log₁₀) in the nasal wash of hamsters after viral challenge on 2 DPI. (d) Comparison of body weight changes for hamsters among Group 1-4 from Day 0 to Day 4 after viral infection. * denotes statistical comparison for mRNA-S+N with mRNA-S (2 μg) and mRNA-S (4 μg). (c) Comparison of viral RNA copies (Log₁₀) in the lung of hamsters on 2 DPI and 4 DPI between Mock, mRNA-S+N, and mRNA-S+N/CD8 depletion groups. (f) Comparison of body weight changes for hamsters between Mock, mRNA-S+N, and mRNA-S+N/CD8 depletion groups. In (f), * denotes statistical comparison for mRNA-S+N with mRNA-S+N/CD8 depletion. One-way ANOVA or student t-test was used for statistical analysis. LOD: limit of detection. * p<0.05, ** p<0.01, *** p<0.001, **** p<0.0001.

FIG. 11. Immune analysis of mRNA-S and combination mRNA-S+N vaccination in mice and hamsters. Three groups of Balb/c mice (7/group) were I.M. vaccinated with mock, mRNA-S (2 μg), or combination mRNA-S+N (2 μg for each) at week 0 and week 3 as indicated above. Blood/serum and splenocytes were collected at indicated times and were subjected to immune analysis. (a-b) ICS measurement of S-specific CD4⁺ and CD8⁺ T cells in the mouse spleen. % individual cytokine-positive, S-specific CD4⁺ (a) or CD8⁺ (b) T cells were examined and compared between the mock and vaccine groups. (c-d) ICS measurement of N-specific CD4⁺ and CD8⁺ T cells in the mouse spleen. % individual cytokine-positive, N-specific CD4⁺ (c) or CD8⁺ (d) T cells were examined and compared between the mock and vaccine groups. (e) IFN-γ ELISPOT measurement of antigen-specific T cells in spleen. Data were shown as SFC #per 10⁶ splenocytes. (f-g) ELISA measurement of serum S-specific (f) or N-specific (g) binding IgG following prime (week 3) or booster (week 5) vaccination in mice. Antibody endpoint titers (EPT) were determined based on serum serial dilutions (1:3 ratio) and were shown for the mock and vaccine groups after prime and booster vaccination. (h) Hamster serum neutralizing activity. Serum samples collected from the hamsters (in FIG. 3b) after booster vaccination (week 5) but prior to viral challenge were measured for neutralizing activity by PRNT. PRNT₅₀ for individual serum samples of each group was shown and compared among different groups as well as between the WT virus and the Delta variant within each group. Dotted line in each plot showed limit of detection (LOD) for each assay. One-way ANOVA or Student's t test were used for statistical analysis. * p<0.05, ** p<0.01, *** p<0.001, **** p<0.0001.

FIG. 12. mRNA-N vaccine design and characterization. (a) Structure of mRNA-N vaccine. Pseudouridine modified RNA encoding full-length SARS-CoV-2 N protein was synthesized, followed by 5′ capping and 3′ poly-A tailing. (b) Western blot confirmation of SARS-CoV-2 N protein expression by mRNA-N. 293T cells were transfected with 2 μg mRNA-N-LNP or PBS for 18 hours. Total protein was extracted from the cells for WB analysis. SARS-CoV-2 N protein was detected using a specific anti-N antibody (MA5-29981).

FIG. 13A-13L. mRNA-N vaccine immunogenicity in mice. (A) Experimental design and timeline. Two groups of BALB/c mice (n=7) were intramuscularly (i.m.) vaccinated with PBS (Mock) or mRNA-N vaccine (1 μg) at weeks 0 and 3. At week 3 before booster vaccination, blood and serum samples were collected for analysis of antibody response. Two weeks after booster vaccination (week 5), mice were euthanized and subjected to immune analysis. (B) Analysis of total CD4 and CD8 T cell activation in the mouse spleen at week 5 after immunization. Expression of CD44 on CD4 and CD8 T cells was examined by flow cytometry and shown as % CD44 of parental population. (C) Vaccine-specific T cells in mouse spleen were measured by ICS. Splenocytes were stimulated with a SARS-CoV-2 N peptide pool (QHD43423.2), followed by immune staining and flow cytometric analysis. Representative flow cytometry plots for cytokine expression in T cells are shown. (D) Shown is the comparison of % cytokine-positive, N-specific CD4 T cells in the spleen between mock and vaccine groups. (E) Shown is the comparison of % cytokine-positive, N-specific CD8 T cells in the spleen between mock and vaccine groups. (F) N-specific T cells in the spleen were measured by IFN-γ ELISPOT. Data were shown as SFC per 10⁶ splenocytes. (G) ELISA measurements are shown for serum N-specific binding IgG following prime (week 3) or booster (week 5) vaccination. OD450 values for individual serum samples after prime or booster vaccination at indicated serum dilution (1:2700 for prime; 1:72900 for booster) are shown. (H) Comparison of N-specific IgG endpoint titers (EPT) between mock and vaccine groups after prime and booster vaccination are shown. (I) Serum neutralizing activity was measured by Plaque Reduction Neutralization Test (PRNT) using wild-type SARS-CoV-2. PRNT₅₀ for individual serum samples of the mock and vaccine groups are shown. Dashed line in (I) indicates the limit of detection. NC, Negative control; PC, positive control. Data are presented as median and IQR. Mann-Whitney (F, G, H) or Kruskal-Wallis (B, D, E) test was used for statistical analysis. ** p<0.01, *** p<0.001. (J) Representative flow cytometry plots are shown for cytokine expression in T cells following dimethyl sulfoxide (DMSO, negative control) or phorbol 12-myristate 13-acetate (PMA)/Ionomycin (positive control) stimulation. TNF-α, tumor necrosis factor-α; IFN-γ, interferon-γ; IL-2, interleukin-2. (K) Representative plots for detecting N-specific T cells in mouse spleen by IFN-γ enzyme-linked immunosorbent spot (ELISPOT) assay are shown. Positive control (anti-CD3 stimulation) and negative control (NC, medium only) for the ELISPOT are also shown. Mock indicates DMSO. (L) Enzyme-linked immunosorbent assay (ELISA) measurement of N-specific binding IgG in serially diluted (1:3) serum samples (n=7 per group). Mean optical density (OD) values (+SD) for serum samples at indicated dilutions after prime (left) and booster (right) vaccination were shown for determination of IgG endpoint titers (EPTs).

FIG. 14A-14F. mRNA-N vaccination induced protection against SARS-CoV-2 challenge in mice and hamsters. (A) Mouse experimental design and timeline. Two groups of BALB/c mice (n=8) were intramuscularly (i.m.) vaccinated with PBS (Mock) or mRNA-N vaccine (1 μg) at week 0 and 3. Two weeks after booster vaccination (week 5), mice were intranasally challenged with mouse-adapted (MA) SARS-CoV-2 (2×10⁴ pfu). Two days post infection (2 DPI), viral loads in the lungs were analyzed to evaluate vaccine-induced protection. (B) Comparison of viral RNA copies in the mouse lungs between mock and vaccine group are shown. Viral RNA copies were quantified by RT-PCR and expressed as Log₁₀ copies per mg of lung tissue. (C) Comparison of viral titers in the mouse lungs between mock and vaccine group are shown. Viral titers were quantified by plaque assay and expressed as Log₁₀ FFU per g of lung tissue. (D) Hamster experimental design and timeline. Three groups of hamsters were investigated. The first two groups (n=12 per group) were i.m. vaccinated with mock or mRNA-N (2 μg) at week 0 and week 3, followed by SARS-CoV-2 Delta challenge at week 5 and viral loads analysis on 2 (n=6) and 4 (n=6) DPI. The third group (n=6) received the same mRNA-N vaccine and subsequent viral challenge, except that these hamsters were intraperitoneally injected with two doses of antibodies for CD8 T cell depletion at six and three days prior to viral challenge. Viral loads were analyzed on 2 DPI (n=6). (E) Comparison of viral RNA copies in hamster lungs (Log₁₀ viral copies/mg) between mock and vaccine group are shown for samples collected on 2 and 4 DPI. (F) Comparison of viral titers in the hamster lungs (Log₁₀ FFU/g) between mock and vaccine group are shown for samples collected on 2 and 4 DPI. (G) Comparison of hamster body weight loss is shown for the mock and vaccine group from Day 0 to 4 DPI. (H) A comparison of viral RNA copies in the lung of hamsters (Log₁₀ viral copies/mg) among the three groups is shown for samples collected on 2 DPI. The dashed line in (F) indicates the limit of detection. Data are presented as median and IQR where appropriate. Mann-Whitney (B, C, G) or Kruskal-Wallis (E, F, H) test was used for statistical analysis. * p<0.05, ** p<0.01, *** p<0.001.

FIG. 15A-15I. Analysis of mRNA-S and mRNA-S+N induced protection in mice against MA-SARS-CoV-2 and in hamsters against Delta. (A) Mouse experimental design and timeline. Three groups of mice (n=8 per group) were vaccinated (i.m.) with mock, mRNA-S (1 μg), or mRNA-S+N (1 μg for each) at week 0 and week 3, followed by intranasal challenge with MA-SARS-CoV-2 (2×10⁴ pfu). On 2 DPI, viral RNA copies and titers in the lungs were quantified. (B) A comparison of viral titers between different groups is shown for mouse lungs collected on 2 DPI (Log₁₀ FFU per g). (C) Shown is a comparison of viral RNA copies in the mouse lungs (Log₁₀ viral copies/mg) between different groups at 2 DPI. (D) Hamster experimental design and timeline. Three groups of hamsters (n=12 per group) were vaccinated (i.m.) with mock, mRNA-S (2 μg), or mRNA-S+N (2 μg for each) at week 0 and week 3, followed by intranasal challenge with SARS-CoV-2 Delta strain (2×10⁴ pfu) at week 5. On 2 (n=6) and 4 DPI (n=6), lung tissues were harvested for analysis of viral RNA copies, viral titers, and pathology; nasal washes were collected for analysis of viral RNA copies; hamster body weights were also monitored. (E) Shown is a comparison of viral titers (Log₁₀ FFU/g) in the hamster lungs between different groups on 2 and 4 DPI. (F) A comparison of viral RNA copies in the hamster lungs (Log₁₀ viral copies/mg) is shown between different groups using samples collected at 2 and 4 DPI. (G) Hamster lung histopathology is shown. Post-challenge lung tissues (2 DPI) were fixed and 5 μm sections cut from hamsters and stained with H&E. Left: Lung of mock-immunized hamsters demonstrates bronchi with bronchiolitis (arrows) and adjacent marked interstitial pneumonia (arrowheads); Middle and Right: Lungs of hamsters immunized with mRNA-S (middle) or mRNA-S+N (right) demonstrate normal bronchial (stars), bronchiolar (arrows), and alveolar architecture. The scale bar for each image indicates 1 mm. (H) A comparison of viral RNA copies in the nasal washes (Log₁₀ viral copies/ml) is shown between the indicated groups on 2 and 4 DPI. (I) A comparison of hamster body weight changes is shown between different groups from Day 0 to Day 4 post infection. * on 2 DPI denotes difference of mRNA-S+N from mRNA-S or mock; ** on 4 DPI denote differences of mRNA-S+N or mRNA-S from mock, respectively. Dashed lines in (B, C, E, and F) show the assay limit of detection. The numbers at the bottom of (B, C, and E) indicate the proportion of animals with a result above the limit of detection. Data are presented as median and IQR where appropriate. Mann-Whitney (B, C) or Kruskal-Wallis (E, F, H, I) test was used for statistical analysis. * p<0.05, ** p<0.01, *** p<0.001.

FIG. 16A-16P. Analysis of mRNA-S and mRNA-S+N induced protection in hamsters against Omicron. (A) Hamster experimental design and timeline. Four groups of hamsters (n=10 per group) were vaccinated (i.m.) with mock (empty LNP), mRNA-S (2 μg), mRNA-S (2 μg), or mRNA-S+N (2 μg for each) at week 0 and week 3, followed by intranasal challenge with SARS-CoV-2 Omicron strain (2×10⁴ pfu) at week 5. On 2 (n=6) and 4 DPI (n=6), lung tissues were harvested for analysis of viral RNA copies, viral titers, and pathology; nasal washes were collected for analysis of viral RNA copies; hamster body weights were monitored. In addition, a fifth group (n=10) that was vaccinated with the same mRNA-S+N but received two doses of anti-CD8β depleting antibody (i.p.) prior to viral challenge (Day-6 and Day-3) was included. (B to E) Viral RNA copies (Log₁₀ viral copies per mg) (B and D) and viral titers (Log₁₀ FFU per g) (C and E) were measured in the hamster lungs collected from the indicated groups at 2 DPI (B and C) and 4 DPI (D and E). (F) Pooled analysis of viral titers are shown for the hamster lung samples collected at 2 DPI and 4 DPI. Log₁₀ FFU/g was compared between the different groups. (G) Hamster lung histopathology is shown using samples collected at 4 DPI. Mock: lung demonstrates bronchi with bronchiolitis (arrows) and adjacent marked interstitial pneumonia (arrowheads); mRNA-S (2 μg): lung demonstrates peribronchiolitis (arrow), perivasculitis (asterisk), and multifocal interstitial pneumonia (arrowhead); mRNA-S (4 μg): lung demonstrates marked interstitial pneumonia (arrowheads); mRNA-S+N: lung demonstrates normal bronchial, bronchiolar (arrows), and alveolar architecture. The scale bar of each image indicates 1 mm. (H) A comparison of viral RNA copies in the nasal washes (Log₁₀ viral copies/ml) between different groups on 2 and 4 DPI is shown. (I) A comparison of hamster body weight changes among the indicated groups is shown. * denotes difference between mRNA-S+N and mRNA-S (4 μg). (J) Shown is a comparison of viral RNA copies in the hamster lungs (Log₁₀ viral copies/mg) on 2 DPI between Mock, mRNA-S+N (S+N), and mRNA-S+N/CD8 T cell depletion (S+N/CD8 Dep) groups. (K) Pooled analysis is shown for viral RNA copies in the hamster lungs for 2 and 4 DPI samples together. (L) Shown is a comparison of hamster body weight changes between the indicated groups. In (L), * and ** denote comparison of mRNA-S+N with mRNA-S+N/CD8 Dep. Dashed lines show limit of detection. The numbers at the bottom of (B to F) indicate the proportion of animals with a result above the limit of detection. Data are presented as median and IQR where appropriate. Kruskal-Wallis test was used for statistical analysis. * p<0.05, ** p<0.01. (M) Lung tissues (2 DPI) were fixed and 5 μm sections cut from mock LNP. mRNA-S (2 μg). mRNA-S (4 μg), or mRNA-S+N-vaccinated hamsters and stained with hematoxylin & cosin (H&E). Lungs of hamsters from all four groups demonstrate normal bronchial, bronchiolar, and alveolar architecture. Scale bar of each image indicates 1 mm. (N) Viral RNA copies (Log₁₀) are shown in the nasal washes of hamsters in different vaccination groups at 4 days after Omicron challenge. Horizontal bars indicate mean. n=5 per group. (O,P) Hamsters were intraperitoneally injected with either mouse anti-Rat CD8β antibody (175 μg, eBio341, functional grade) or PBS as control on Day-6 and Day-3, as described in the Methods. Three days after second antibody injection (Day 0), splenocytes were isolated from the hamsters and stained with anti-CD8β-phycoerythrin (PE) (clone: eBio341). The percentage of CD8⁺ T cells in splenocytes was examined by flow cytometry. (O) Shown are representative flow cytometry plots for CD8 staining in splenocytes of two control (Ctrl, top) and two CD8-Depleted (CD8-Dep, bottom) hamsters. % CD8⁺ (or CD8^{hi}) in splenocytes are shown. (P) Depletion efficiency was expressed as % CD8⁺ T cells in splenocytes of the depleted hamsters relative to that of control hamsters (77% depletion). Data are presented as mean±SD.

FIG. 17A-17I. Combination mRNA-S+N vaccination induces antigen-specific immune responses in mice and hamsters. Immunogenicity experimental design and timeline: three groups of BALB/c mice (n=7 per group) were vaccinated (i.m.) with mock, mRNA-S (1 μg), or combination mRNA-S+N (1 μg for each) at week 0 and week 3. Blood and serum samples were collected at week 3 (prior to booster) to measure antibody responses. Two weeks after booster (week 5), vaccine-induced T cell and antibody responses were measured (A to G). (A and B) ICS measurements of S-specific CD4 and CD8 T cells in the mouse spleen (week 5) are shown. % individual cytokine-positive CD4 (A) or CD8 (B) T cells were compared between the mock and vaccine groups. (C and D) ICS measurement of N-specific CD4 and CD8 T cells in the mouse spleen (week 5) are shown. % individual cytokine-positive CD4 (C) or CD8 (D) T cells were compared between the mock and vaccine groups. (E) IFN-γ ELISPOT measurements of antigen-specific T cells in spleen (week 5) are shown. Data were shown as SFC per 10⁶ splenocytes. (F and G) ELISA measurement of serum S-specific (F) or N-specific (G) binding IgG are shown for samples collected following prime (week 3) or booster (week 5) vaccination in mice. Antibody endpoint titers (EPTs) were determined based on serum serial dilutions and compared between different groups. (H) Serum samples were collected from the hamsters (n=12) after booster vaccination (week 5) but prior to viral challenge. Samples were used to measure neutralizing activity by PRNT. PRNT₅₀ neutralization titers for individual serum samples were compared among different groups as well as between the wild-type virus and the Delta variants. Dashed lines (F, G, H) show limit of detection for each assay. Data are presented as median and IQR where appropriate. Kruskal-Wallis test was used for statistical analysis. * p<0.05, ** p<0.01, *** p<0.001, **** p<0.0001. (I) Shown are representative IFN-γ ELISPOT for detecting S-specific and N-specific T cells in the mouse spleen following mock, mRNA-S, or combination mRNA-S+N vaccination. Positive control (anti-CD3 stimulation) and negative control (medium only) for the IFN-γ ELISPOT are shown.

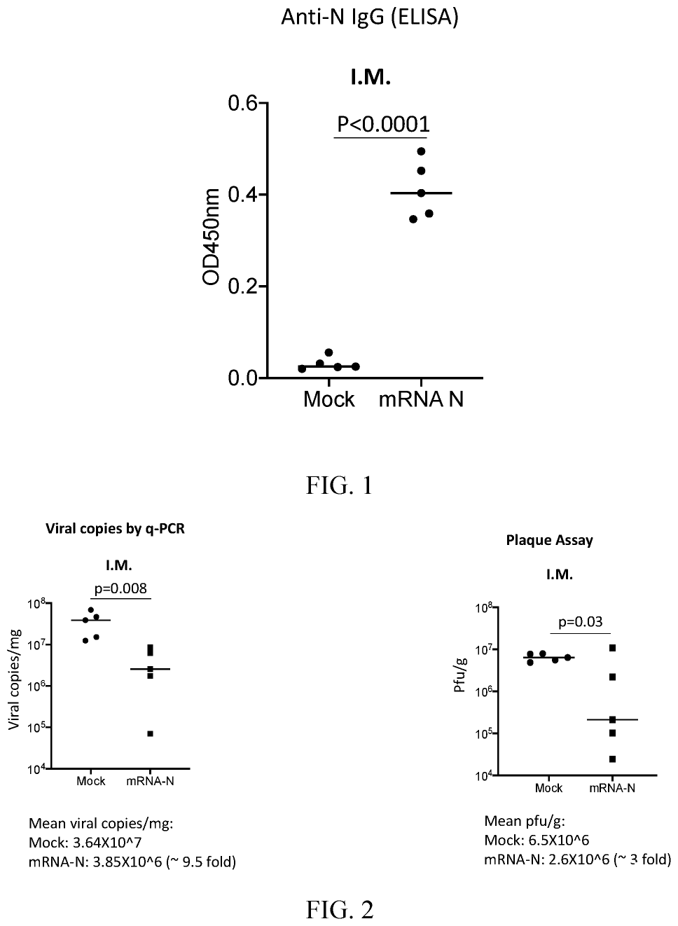

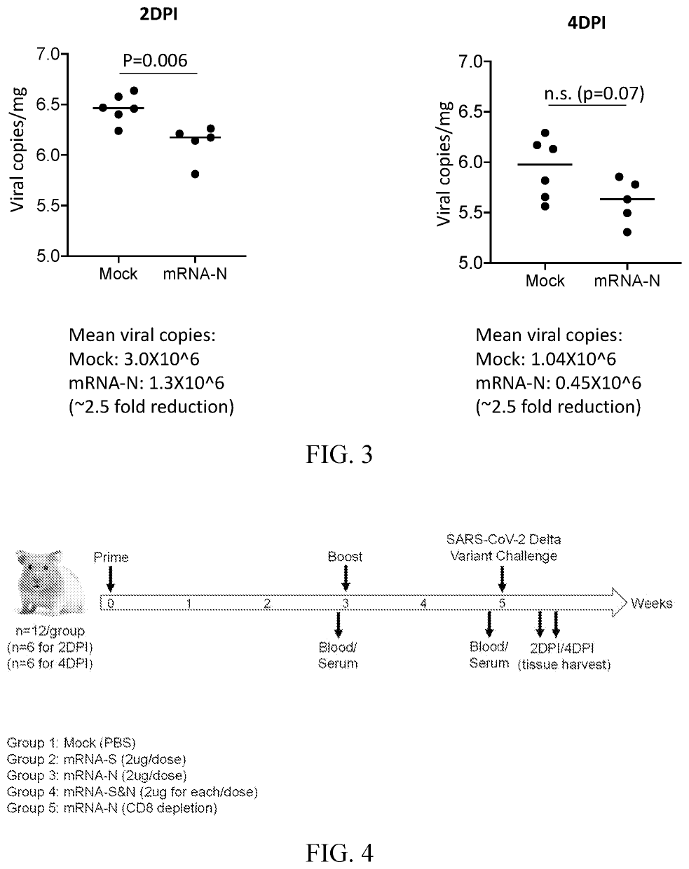

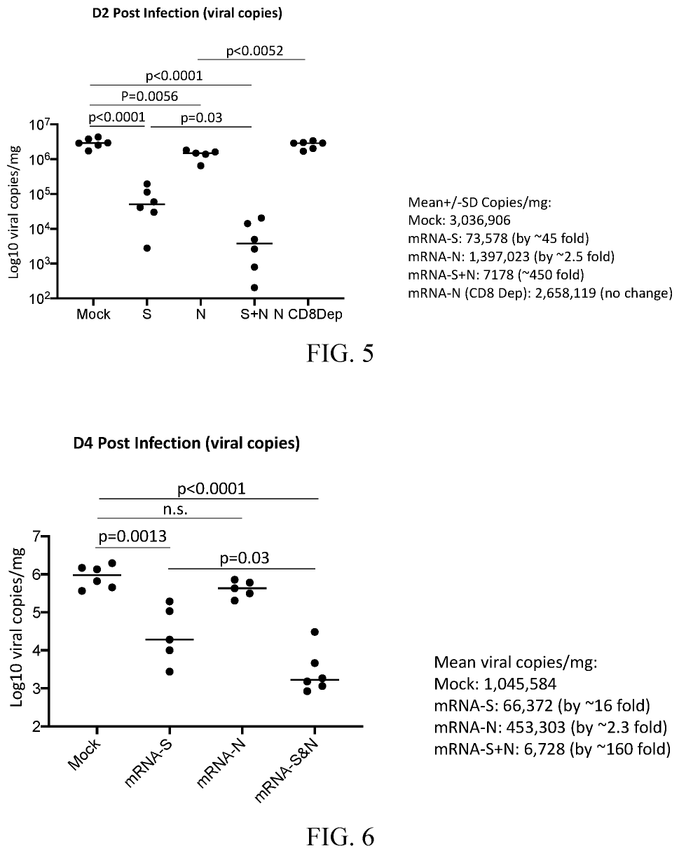

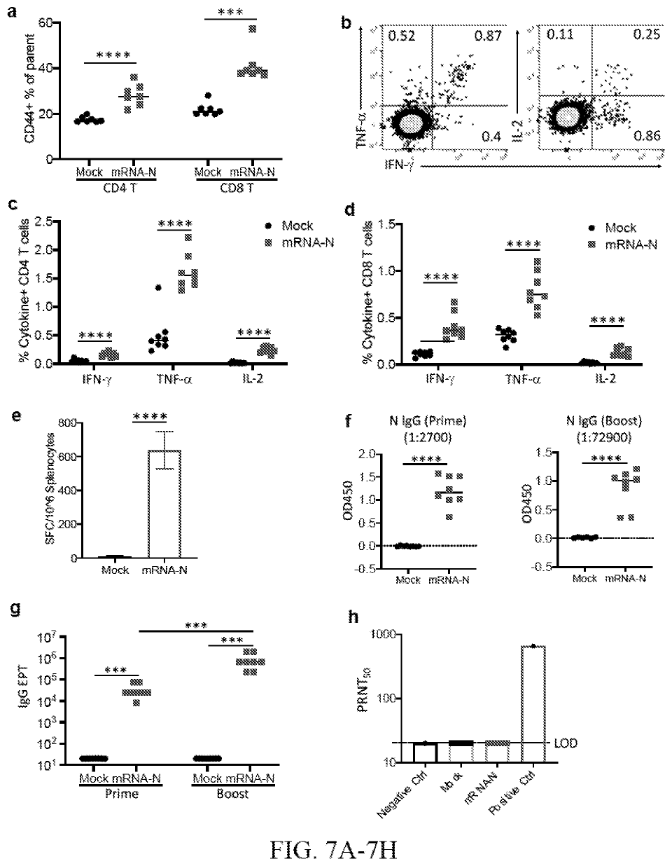

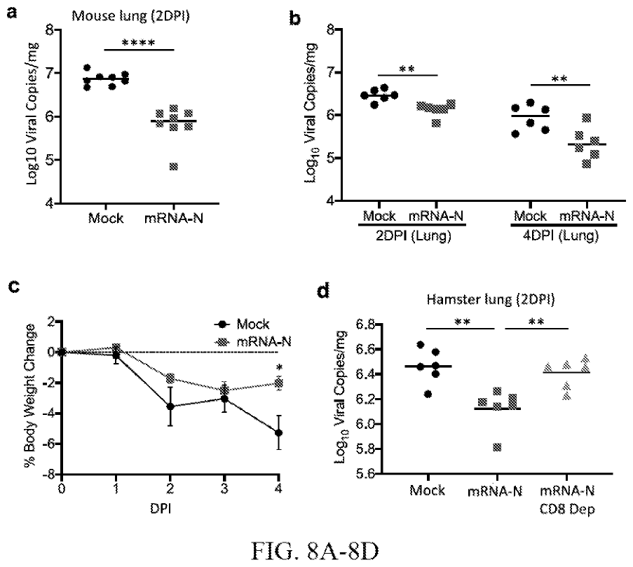

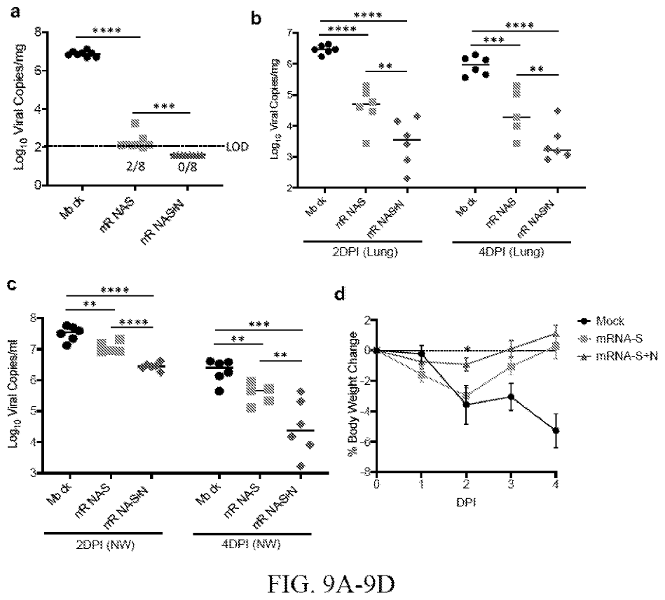

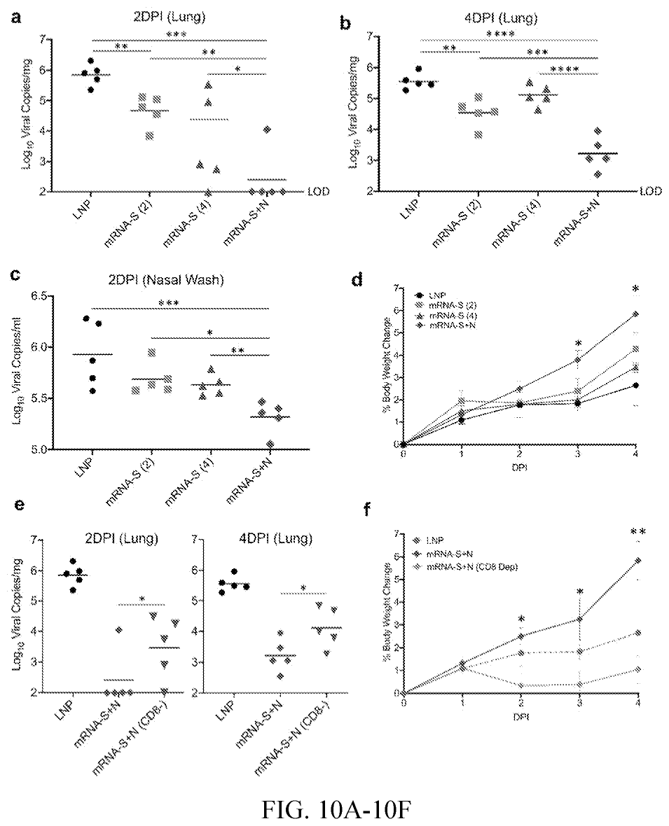

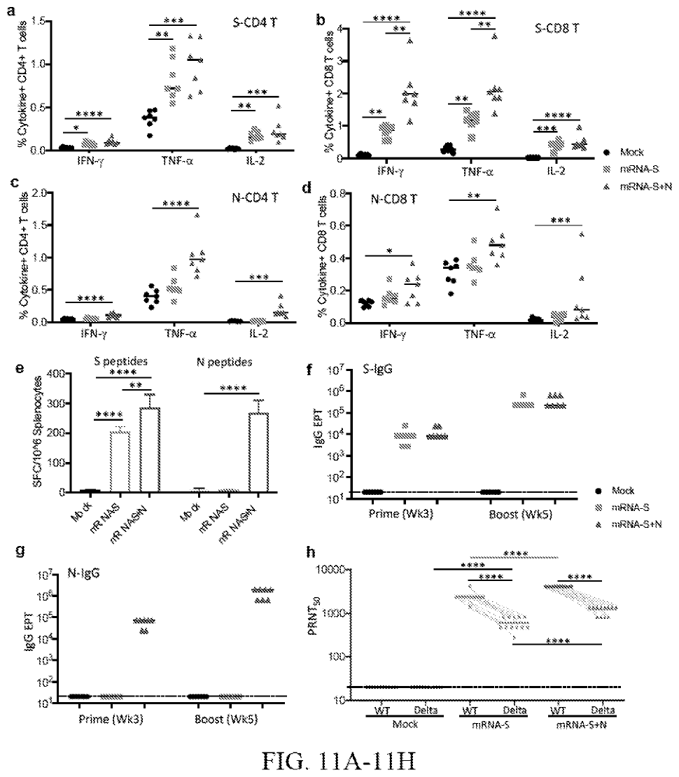

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to intimate that the scope of the disclosure, including the claims, is limited to that embodiment.

A current interest in the fields of therapeutics and diagnostics is the ability and methods for designing, synthesizing, and delivering a nucleic acid to effect physiologic outcomes beneficial to a cell, a tissue, an organ, and ultimately to a subject. The nucleic acid can be a ribonucleic acid (RNA) such as a messenger RNA (mRNA) encoding a peptide or polypeptide of interest. One beneficial outcome is the intracellular translation of the nucleic acid and production of at least one encoded peptide or polypeptide of interest. Of particular interest, is the ability to design, synthesize and deliver a nucleic acid, such as a ribonucleic acid (RNA) which encodes an antigen for the purpose of vaccination.

Described herein are compositions (including pharmaceutical compositions) and methods for the design, preparation, manufacture, formulation, and/or use of nucleic acid vaccines (NAVs) where at least one component of the NAV is a nucleic acid molecule. In particular, described herein are compositions (including pharmaceutical compositions) and methods for the selection, design, preparation, manufacture, formulation, and/or use of nucleic acid vaccines (NAVs) where at least one component of the NAV is a polynucleotide, a RNA polynucleotide, and/or a mRNA which encodes an antigen derived from an infectious microorganism, in particular SARS-CoV-2. Also provided are systems, processes, devices and kits for the selection, design and/or utilization of the NAVs described herein. Certain embodiments are directed to a DNA encoding such a mRNA vaccine that can be used to produce and manufacture the mRNA vaccine, including a producer cell line hosting the vaccine encoding DNA.

SARS-CoV-2 can cause severe respiratory disease in humans. The SARS CoV-2 viral spike(S) protein binds to angiotensin-converting enzyme 2 (ACE2), which is the entry receptor utilized by SARS-CoV-2. The spike(S) protein of coronaviruses is a major surface protein and is a target for neutralizing antibodies in infected patients (Lester et al., Access Microbiology 2019; 1); and is therefore considered a potential protective antigen for vaccine design.

The SARS-CoV N protein contains two distinct RNA-binding domains (the N-terminal domain [NTD] and the C-terminal domain [CTD]) linked by a poorly structured linkage region (LKR) containing a serine/arginine-rich (SR-rich) domain (SRD). Due to the positive amino acids, SARS-CoV N-NTD and N-CTD have been reported to bind with viral RNA genome. LKR is able to improve oligomerization. However, the molecular properties of SARS-CoV-2 N protein remain to be fully defined.

An additional Coronavirus vaccine is described herein as an mRNA vaccine encoding a SARS-CoV-2 nucleoprotein (mRNA-N), as well as a SARS-CoV-2 spike protein(S) (mRNA-Spp) variant. Certain embodiments employ an mRNA-N, mRNA-S, or mRNA-N and mRNA-S vaccine to vaccinate a subject against SARS-CoV-2.

Studies have shown that mRNA-N alone induces modest but significant suppression of SARS-CoV-2 delta variant in hamster (˜2-2.5 fold). The suppressive effect of mRNA-N on SARS-CoV-2 is confirmed in a mouse model (using mouse-adapted SARS-CoV-2 strain). mRNA-S alone (S2P) induces strong suppression of delta variant (2 DPI: ˜45 fold reduction). Combinatorial mRNA vaccination (N/S) has demonstrated synergistic effects and substantially enhances suppression (by ˜450 fold).

### I. Nucleic Acid Vaccines (NAVs)

Nucleic Acid Vaccines (NAVs) described herein comprise one or more polynucleotides (platform or construct) which encode one or more Coronavirus antigens. Polynucleotide constructs include antigen-encoding RNA polynucleotides such as mRNAs. The polynucleotide constructs can include at least one chemical modification. The sequences provided can be the sense strand of a sequence but one of skill would readily identify the complementary anti-sense sequence as well. Also, the nucleotide sequences may be presented as DNA sequences, deoxyribose adenine, guanine, thymine, cytosine (AGTC) and/or RNA sequences ribose adenine, guanine, uracil, cytosine (AGUC); one of skill would readily identify the RNA or DNA counterpart.

NAV compositions of the invention may comprise other components including, but not limited to, adjuvants. Adjuvants may also be administered with or in combination with one or more NAVs. In one aspect, an adjuvant acts as a co-signal to prime T-cells and/or B-cells and/or NK cells as to the existence of an infection. Adjuvants may be co-administered by any route, e.g., intramusculary, subcutaneous, IV or intradermal injections. Adjuvants useful in the present invention may include, but are not limited to, natural or synthetic adjuvants. Adjuvants can be selected from any of the classes (1) mineral salts, e.g., aluminium hydroxide and aluminium or calcium phosphate gels; (2) emulsions including: oil emulsions and surfactant based formulations, e.g., microfluidized detergent stabilized oil-in-water emulsion, purified saponin, oil-in-water emulsion, stabilized water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; (6) inert vehicles, such as gold particles; (7) microorganism derived adjuvants; (8) tensoactive compounds; (9) carbohydrates; or combinations thereof.

Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA 1-DD gene fusion protein, DOC/Alum Complex, Gamma Inulin, Gerbu Adjuvant, GM-CSF, GMDP, Recombinant hIFN-gamma/Interferon-g, Interleukin-1B, Interleukin-2, Interleukin-7, Sclavo peptide, Rchydragel LV, Rehydragel HPA, Loxoribine, MF59, MTP-PE Liposomes, Murametide. Murapalmitine, D-Murapalmitinc, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR1920 Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant. AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and/or Matrix-S. Other adjuvants which may be co-administered with the NAVs of the invention include, but are not limited to interferons, TNF-alpha, TNF-beta, chemokines such as CCL21, cotaxin, HMGB1, SA100-8alpha, GCSF, GMCSF, granulysin, lactoferrin, ovalbumin, CD-40L, CD28 agonists, PD-1, soluble PD1, L1 or L2, or interleukins such as IL-1, IL-2, IL-4, IL-6, IL-7, IL-10. IL-12, IL-13, IL-21. IL-23, IL-15, IL-17, and IL-18. These may be administered with the NAV on the same encoded polynucleotide, e.g., polycistronic, or as separate mRNA encoding the adjuvant and antigen.

NAVs of the present invention may vary in their valency. Valency refers to the number of antigenic components in the NAV polynucleotide. In some embodiments, the NAVs are monovalent (monocistronic). In some embodiments, the NAVs are divalent (bicistronic). The antigenic components of the NAVs may be on a single polynucleotide or on separate polynucleotides.

An “effective amount” of the NAV composition is provided based, at least in part, on the target tissue, target cell type, means of administration, physical characteristics of the polynucleotide (e.g., size, and extent of modified nucleosides) and other components of the NAV, and other determinants. In general, an effective amount of the NAV composition provides an induced or boosted immune response as a function of antigen production in the cell.

Activation of the Immune Response. According to various embodiments, the NAVs comprising the polynucleotides disclosed herein may act as a vaccine. As used herein, a “vaccine” refers to a composition, for example, a substance or preparation that stimulates, induces, causes or improves immunity in an organism, e.g., a mammalian organism (a human, etc.). Preferably, a vaccine provides immunity against one or more diseases or disorders, including prophylactic and/or therapeutic immunity. NAVs may be administered prophylactically or therapeutically as part of an active immunization scheme to healthy individuals or early in infection during the incubation phase or during active infection after onset of symptoms.

The use of RNA in or as a vaccine overcomes the disadvantages of conventional genetic vaccination involving incorporating DNA into cells in terms of safeness, feasibility, applicability, and effectiveness to generate immune responses. RNA molecules are considered to be significantly safer than DNA vaccines, as RNAs are more easily degraded. They are cleared quickly out of the organism and cannot integrate into the genome and influence the cell's gene expression in an uncontrollable manner. It is also less likely for RNA vaccines to cause severe side effects like the generation of autoimmune disease or anti-DNA antibodies (Bringmann et al., Journal of Biomedicine and Biotechnology, 2010). Transfection with RNA requires only insertion into the cell's cytoplasm, which is easier to achieve than into the nucleus. However, RNA is susceptible to RNase degradation and other natural decomposition in the cytoplasm of cells. In certain aspects, a mRNA vaccine is configured to express the encoded polypeptide in a cell of a vaccinated subject.

In one embodiment, the polynucleotides of the NAVs of the invention may be administrated with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, or more.

In certain aspects, the polynucleotides of the NAVs of the invention may be administered intranasally, intramuscularly, or intradermally.

In certain aspects, NAVs can be used as memory booster vaccines and are administered to boost antigenic memory across a time period of 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more years.

### II. NAV Polynucleotides

According to certain embodiments, the polynucleotides encode at least one polypeptide of interest (an antigen or immunogen). Antigens of the present invention may be wild type derived from Coronavirus or modified, engineered, designed or artificial. They may have any combination of the features described herein. In certain embodiments, the antigen is derived from the S protein, N protein, or the S protein and N protein of a Coronaviurs (e.g., SARS-CoV-2).

Certain embodiments are directed to nucleic acid molecules that encode one or more peptides or polypeptides of interest. Such peptides or polypeptides serve as an antigen or antigenic molecule. The term “nucleic acid,” in its broadest sense, includes any compound and/or substance that comprise a polymer of nucleotides. These polymers are often referred to as polynucleotides. Nucleic acids or polynucleotides of the invention include, but are not limited to, ribonucleic acids (RNAs), which may or may not include ribonucleotide analogs or modifications. In some embodiments, polynucleotides of the present disclosure is or functions as a messenger RNA (mRNA). As used herein the term “messenger RNA” (mRNA) refers to any polynucleotide that encodes at least one polypeptide (a naturally-occurring, non-naturally-occurring, or modified polymer of amino acids) and can be translated to produce the encoded polypeptide in vitro, in vivo, in situ, or ex vivo.

In certain aspects, the polynucleotides of the present invention that are circular are known as “circular polynucleotides” or “circP.” As used herein, “circular polynucleotides” or “circP” means a single stranded circular polynucleotide which acts substantially like, and has the properties of, an RNA. The term “circular” is also meant to encompass any secondary or tertiary configuration of the circP.

In one embodiment, the length of a region encoding at least one polypeptide of interest of the polynucleotides present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000 nucleotides). As used herein, such a region may be referred to as a “coding region” or “region encoding” or “open reading frame (ORF)”.

In one embodiment, the polynucleotides of the present invention is or functions as a messenger RNA (mRNA). As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo.

In one embodiment, the polynucleotides of the present invention may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In certain aspects, the polynucleotides have a uniform chemical modification of all or any of the same nucleoside type or a population of modifications, or a measured percent of a chemical modification of all any of the same nucleoside type but with random incorporation, such as where all uridines are replaced by a uridine analog, e.g., pseudouridine. In another embodiment, the polynucleotides may have a uniform chemical modification of two, three, or four of the same nucleoside type throughout the entire polynucleotide (such as all uridines and all cytosines, etc. are modified in the same way).

When the polynucleotides of the present invention are chemically and/or structurally modified the polynucleotides may be referred to as “modified polynucleotides.”

Polynucleotide Architecture. Traditionally, the basic components of an mRNA molecule include at least a coding region, a 5′UTR, a 3′UTR, a 5′ cap and a poly-A tail. The polynucleotides described herein may function as mRNA. Embodiments are directed to an mRNA having a coding region and one or more of a 5′UTR, a 3′UTR, a 5′ cap and a poly-A tail. The polynucleotides described herein may function as mRNA.

Circular Polynucleotide Architecture. Certain aspects are directed to polynucleotides which are circular or cyclic. As the name implies circular polynucleotides are circular in nature meaning that the termini are joined in some fashion, whether by ligation, covalent bond, common association with the same protein or other molecule or complex or by hybridization. The circular polynucleotides or circPs that encode at least one peptide or polypeptide of interest are known as circular RNAs or circRNA. The antigens of the NAVs of the present invention may be encoded by one or more circular RNAs or circRNAs. As used herein, “circular RNA” or “circRNA” means a circular polynucleotide that can encode at least one peptide or polypeptide of interest.

As used herein, “polypeptide” means a polymer of amino acid residues (natural or unnatural) linked together most often by peptide bonds. The term, as used herein, refers to proteins, polypeptides, and peptides of any size, structure, or function. In one embodiment, the polypeptides of interest are antigens encoded by the polynucleotides as described herein.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue.

Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to polypeptides are those with one or more amino acids in the native or starting amino acid sequence removed. Ordinarily, deletional variants will have one or more amino acids deleted in a particular region of the molecule.

“Covalent derivatives” when referring to polypeptides include modifications of a native or starting protein with an organic proteinaccous or non-proteinaceous derivatizing agent, and/or post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

In some embodiments, the encoded polypeptide variant may have the same or a similar activity as the reference polypeptide (e.g., S or N proteins). Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) Other tools are described herein, specifically in the definition of “Identity.”

Default parameters in the BLAST algorithm include, for example, an expect threshold of 10, Word size of 28, Match/Mismatch Scores 1, −2, Gap costs Linear. Any filter can be applied as well as a selection for species specific repeats, e.g., Homo sapiens.

Cell-Penetrating Polypeptides. The polynucleotides disclosed herein may also encode one or more cell-penetrating polypeptides. As used herein, “cell-penetrating polypeptide” or CPP refers to a polypeptide which may facilitate the cellular uptake of molecules. A cell-penetrating polypeptide of the present invention may contain one or more detectable labels. The polypeptides may be partially labeled or completely labeled throughout. The polynucleotides may encode the detectable label completely, partially or not at all. The cell-penetrating peptide may also include a signal sequence. As used herein, a “signal sequence” refers to a sequence of amino acid residues bound at the amino terminus of a nascent protein during protein translation. The signal sequence may be used to signal the secretion of the cell-penetrating polypeptide.

In one embodiment, the polynucleotides may also encode a fusion protein. The fusion protein may be created by operably linking a heterologous protein or peptide to a therapeutic protein. As used herein, “operably linked” refers to the therapeutic protein and the heterologous protein or peptide being connected in such a way to permit the expression of the complex when introduced into the cell. Preferably, the therapeutic protein may be covalently linked to the heterologous protein or peptide in the formation of the fusion protein.

Polynucleotides Having Untranslated Regions (UTRs). The polynucleotides of the present invention (e.g., antigen-encoding polynucleotides featured in the NAVs of the invention) may comprise one or more regions or parts which act or function as an untranslated region. Where polynucleotides are designed to encode at least one polypeptide of interest, the polynucleotides may comprise one or more of these untranslated regions.

By definition, untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. The regulatory features of UTR can be incorporated into the polynucleotides of the present invention to among other things, enhance the stability of the molecule.

Natural 5′UTRs bear features which play roles in translation initiation. They harbor signatures like Kozak sequences which are commonly known to be involved in the process by which the ribosome initiates translation of many genes. 5′UTR also have been known to form secondary structures which are involved in elongation factor binding. By engineering the features typically found in abundantly expressed genes of specific target organs, one can enhance the stability and protein production of the polynucleotides of the invention.

Other non-UTR sequences may also be used as regions or subregions within the polynucleotides. For example, introns or portions of introns sequences may be incorporated into regions of the polynucleotides of the invention. Incorporation of intronic sequences may increase protein production as well as polynucleotide levels.

Combinations of features may be included in flanking regions and may be contained within other features. For example, the ORF may be flanked by a 5′ UTR which may contain a strong Kozak translational initiation signal and/or a 3′ UTR which may include an oligo (dT) sequence for templated addition of a poly-A tail. 5′UTR may comprise a first polynucleotide fragment and a second polynucleotide fragment from the same and/or different genes.

A UTR from various gene(s) may be incorporated into the regions of the polynucleotide. Furthermore, multiple UTRs of any known gene may be utilized. It is also within the scope of the present invention to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In one embodiment, flanking regions are selected from a family of transcripts whose proteins share a common function, structure, feature of property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

3′ UTR and the AU Rich Elements. Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. C-Myc and MyoD contain class I AREs. Class II AREs possess two or more overlapping UUAUUUA (U/A) (U/A) nonamers. Molecules containing this type of AREs include GM-CSF and TNF-α. Class III ARES are less well defined. These U rich regions do not contain an AUUUA motif. c-Jun and Myogenin are two well-studied examples of this class.

Regions Having a 5′ Cap. The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or ante-terminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

In some embodiments, polynucleotides may be designed to incorporate a cap moiety. Modifications to the polynucleotides of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide which functions as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which may equivalently be designated 3′ O-Me-m7G (5′) ppp (5′) G). The 3′-0 atom of the other, unmodified, guanine becomes linked to the 5′-terminal nucleotide of the capped polynucleotide. The N7- and 3′-O-methlyated guanine provides the terminal moiety of the capped polynucleotide.

Another example of a cap is mCAP, which is similar to ARCA but has a 2′-O-methyl group on guanosine (i.e., N7,2′-O-dimethyl-guanosine-5′-triphosphate-5′-guanosine, m7Gm-ppp-G).

Viral Sequences. Additional viral sequences such as, but not limited to, the translation enhancer sequence of the barley yellow dwarf virus (BYDV-PAV), the Jaagsiekte sheep retrovirus (JSRV) and/or the Enzootic nasal tumor virus (See e.g., International Pub. No. WO2012129648; herein incorporated by reference in its entirety) can be engineered and inserted in the polynucleotides of the invention and can stimulate the translation of the construct in vitro and in vivo. Transfection experiments can be conducted in relevant cell lines at and protein production can be assayed by ELISA at 12 hr, 24 hr, 48 hr, 72 hr and day 7 post-transfection.

IRES Sequences. Further, provided are polynucleotides (e.g., antigen-encoding polynucleotides featured in the NAVs of the invention) which may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. Polynucleotides containing more than one functional ribosome binding site may encode several peptides or polypeptides that are translated independently by the ribosomes (“multicistronic nucleic acid molecules”). When polynucleotides are provided with an IRES, further optionally provided is a second translatable region. Examples of IRES sequences that can be used according to the invention include without limitation, those from coxsackievirus B3 (CVB3), picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

Poly-A Tails. During RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

According to the present invention, terminal groups on the poly A tail may be incorporated for stabilization into polynucleotides of the invention (e.g., antigen-encoding polynucleotides featured in the RNAVs of the invention). Polynucleotides of the present invention may include des-3′ hydroxyl tails. They may also include structural moieties or 2′-Omethyl modifications as taught by Junjie Li, et al. (Current Biology, Vol. 15, 1501-1507, 2005).

The polynucleotides may be designed to encode transcripts with alternative polyA tail structures including histone mRNA. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs

Unique poly-A tail lengths provide certain advantages to the polynucleotides of the present invention (e.g., antigen-encoding polynucleotides featured in the NAVs of the invention).

Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1.500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1.000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall polynucleotide or the length of a particular region of the polynucleotide. This design may be based on the length of a coding region, the length of a particular feature or region or based on the length of the ultimate product expressed from the polynucleotides.

In this context the poly-A tail may be 10, 20, 30, 40, 50, 60, 70, 80, 90, or 100% greater in length than the polynucleotide or feature thereof. The poly-A tail may also be designed as a fraction of the polynucleotides to which it belongs. In this context, the poly-A tail may be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein may enhance expression.

Start Codon Region. In some aspects, the polynucleotides may have regions that are analogous to or function like a start codon region. In one embodiment, the translation of a polynucleotide may initiate on a codon which is not the start codon AUG. Translation of the polynucleotide may initiate on an alternative start codon such as, but not limited to, ACG, AGG, AAG, CTG/CUG, GTG/GUG, ATA/AUA, ATT/AUU, TTG/UUG. As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See e.g., Matsuda and Mauro PLOS ONE, 2010 5:11; the contents of which are herein incorporated by reference in its entirety). Masking any of the nucleotides flanking a codon that initiates translation may be used to alter the position of translation initiation, translation efficiency, length and/or structure of a polynucleotide.

Stop Codon Region. In one aspect, the polynucleotides may include at least one or two stop codons before the 3′ untranslated region (UTR). The stop codon may be selected from TGA, TAA and TAG. In one aspect, the polynucleotides include the stop codon TGA and one additional stop codon. In a further embodiment the addition stop codon may be TAA. In another embodiment, the polynucleotides of the present invention include three stop codons.

Signal Sequences. The polynucleotides described herein may also encode additional features which facilitate trafficking of the polypeptides to therapeutically relevant sites. One such feature which aids in protein trafficking is the signal sequence. As used herein, a “signal sequence” or “signal peptide” is a polynucleotide or polypeptide, respectively, which is from about 9 to 200 nucleotides (3-60 amino acids) in length which is incorporated at the 5′ (or N-terminus) of the coding region or polypeptide encoded, respectively. Addition of these sequences result in trafficking of the encoded polypeptide to the endoplasmic reticulum through one or more secretory pathways. Some signal peptides are cleaved from the protein by signal peptidase after the proteins are transported.

Protein Cleavage Signals and Sites. In certain aspects, polypeptides of the invention (e.g., antigen polypeptides) may include various protein cleavage signals and/or sites.

In one embodiment, the polypeptides of the present invention may include at least one protein cleavage signal containing at least one protein cleavage site. The protein cleavage site may be located at the N-terminus, the C-terminus, at any space between the N- and the C-termini such as, but not limited to, half-way between the N- and C-termini, between the N-terminus and the half-way point, between the half-way point and the C-terminus, and combinations thereof.

In one embodiment, the polynucleotides of the present invention may be engineered such that the polynucleotide contains at least one encoded protein cleavage signal. The encoded protein cleavage signal may be located in any region including but not limited to before the start codon, after the start codon, before the coding region, within the coding region such as, but not limited to, half way in the coding region, between the start codon and the half way point, between the half way point and the stop codon, after the coding region, before the stop codon, between two stop codons, after the stop codon and combinations thereof.

In one embodiment, the polynucleotides of the present invention may include at least one encoded protein cleavage signal containing at least one protein cleavage site. The encoded protein cleavage signal may include, but is not limited to, signalase cleavage signal (SEQ ID NO: 10), a proprotein convertase (or prohormone convertase), thrombin and/or Factor Xa protein cleavage signal.

Codon Optimization. The polynucleotides contained in the NAVs of the invention, their regions or parts or subregions may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park Calif.) and/or proprietary methods. In one embodiment, the ORF sequence is optimized using optimization algorithms.

In some embodiments, a 5′ UTR and/or a 3′ UTR region may be provided as flanking regions. Multiple 5′ or 3′ UTRs may be included in the flanking regions and may be the same or of different sequences. Any portion of the flanking regions, including none, may be codon optimized and any may independently contain one or more different structural or chemical modifications, before and/or after codon optimization.

In Vitro Transcription-Enzymatic Synthesis. cDNA encoding the polynucleotides described herein may be transcribed using an in vitro transcription (IVT) system. The system typically comprises a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate polynucleotides (e.g., modified nucleic acids).

Solid-Phase Chemical Synthesis. Chimeric polynucleotides or circular polynucleotides described herein may be manufactured in whole or in part using solid phase techniques.

Solid-phase chemical synthesis of polynucleotides or nucleic acids is an automated method wherein molecules are immobilized on a solid support and synthesized step by step in a reactant solution. Impurities and excess reagents are washed away and no purification is required after each step. The automation of the process is amenable on a computer-controlled solid-phase synthesizer. Solid-phase synthesis allows rapid production of polynucleotides or nucleic acids in a relatively large scale that leads to the commercial availability of some polynucleotides or nucleic acids. Furthermore, it is useful in site-specific introduction of chemical modifications in the polynucleotide or nucleic acid sequences. It is an indispensable tool in designing modified derivatives of natural nucleic acids.

Liquid Phase Chemical Synthesis. The synthesis of chimeric polynucleotides or circular polynucleotides of the present invention (e.g., antigen-encoding polynucleotides featured in the NAVs of the invention) by the sequential addition of monomer building blocks may be carried out in a liquid phase. A covalent bond is formed between the monomers or between a terminal functional group of the growing chain and an incoming monomer. Functional groups not involved in the reaction must be temporarily protected. After the addition of each monomer building block, the reaction mixture has to be purified before adding the next monomer building block. The functional group at one terminal of the chain has to be deprotected to be able to react with the next monomer building blocks. A liquid phase synthesis is labor- and time-consuming and cannot not be automated. Despite the limitations, liquid phase synthesis is still useful in preparing short polynucleotides in a large scale. Because the system is homogenous, it does not require a large excess of reagents and is cost-effective in this respect.

Combination of Synthetic Methods. The synthetic methods discussed above each has its own advantages and limitations. Attempts have been conducted to combine these methods to overcome the limitations. Such combinations of methods are within the scope of the present invention.

### III. Modifications

In certain embodiments, polynucleotides described herein can include various substitutions and/or insertions. As used herein the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population. Generally, herein, these terms are not intended to refer to the ribonucleotide modifications in naturally occurring 5′-terminal mRNA cap moieties. In a polypeptide, the term “modification” refers to a modification as compared to the canonical set of 20 amino acids.

The modifications may be various distinct modifications. In some embodiments, the regions may contain one, two, or more (optionally different) nucleoside or nucleotide modifications. In some embodiments, a modified polynucleotide, introduced to a cell may exhibit reduced degradation in the cell, as compared to an unmodified polynucleotide.

The polynucleotides of the NAVs of the invention can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Non-natural modified nucleotides may be introduced to polynucleotides during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme.

Modified Polynucleotide Molecules. The present invention also includes building blocks, e.g., modified ribonucleosides, and modified ribonucleotides, of polynucleotide molecules, e.g., of the NAVs of the invention. For example, these building blocks can be useful for preparing the polynucleotides of the invention.

Modifications on the Sugar. The modified nucleosides and nucleotides which may be incorporated into a polynucleotide can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C1-6 alkyl: optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein); a polyethyleneglycol (PEG), —O(CH₂CH₂O)nCH₂CH₂R, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se. or alkylene, such as methylene or ethylene); addition of a double bond (e.g., to replace ribose with cyclopentenyl or cyclohexenyl); ring contraction of ribose (e.g., to form a 4-membered ring of cyclobutane or oxctane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′->2′)), and peptide nucleic acid (PNA, where 2-amino-ethyl-glycine linkages replace the ribose and phosphodiester backbone). The sugar group can also contain one or more carbons that possess the opposite stercochemical configuration than that of the corresponding carbon in ribose. Thus, a polynucleotide molecule can include nucleotides containing, e.g., arabinose, as the sugar. Such sugar modifications are taught International Application Number PCT/2012/058519 filed Oct. 3, 2012 (Attorney Docket Number M9) and U.S. Provisional Application No. 61/837,297 filed Jun. 20, 2013 (Attorney Docket Number M36) the contents of each of which are incorporated herein by reference in its entirety.

Modifications on the Nucleobase. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphoester linkages, in which case the polynucleotides would comprise regions of nucleotides.

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. Such modified nucleobases (including the distinctions between naturally occurring and non-naturally occurring) are taught in International Application Number PCT/2012/058519 filed Oct. 3, 2012 (Attorney Docket Number M9) and U.S. Provisional Application No. 61/837,297 filed Jun. 20, 2013 (Attorney Docket Number M36) the contents of each of which are incorporated herein by reference in its entirety.

### IV. Pharmaceutical Vaccine Compositions

The present invention provides pharmaceutical compositions including NAVs and NAV compositions and/or complexes optionally in combination with one or more pharmaceutically acceptable excipients. The present invention provides NAVs and NAV pharmaceutical compositions and complexes optionally in combination with one or more pharmaceutically acceptable excipients. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21′ ed., Lippincott Williams & Wilkins, 2005 (incorporated herein by reference in its entirety).

In some embodiments, compositions are administered to humans, human patients or subjects. For the purposes of the present disclosure, the phrase “active ingredient” generally refers to the NAVs or the polynucleotides contained therein, e.g., antigen-encoding polynucleotides, for example, RNA polynucleotides, to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%. e.g., between 0.5 and 50%, between 1-30%, between 5-80%, at least 80% (w/w) active ingredient.

Formulations. The NAVs of the invention can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with NAVs (e.g., for transplantation into a subject), hyaluronidase, nanoparticle mimics and combinations thereof.

Accordingly, the formulations of the invention can include one or more excipients, each in an amount that may increases the stability of the NAV, increases cell transfection by the NAV, increases the expression of polynucleotides encoded protein, and/or alters the release profile of polynucleotide encoded proteins. Further, the polynucleotides of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one polynucleotide, e.g., antigen-encoding polynucleotide. As a non-limiting example, the formulations may contain 1, 2, 3, 4 or 5 polynucleotides.

In one embodiment, the formulations described herein may comprise more than one type of polynucleotide, e.g., antigen-encoding polynucleotide. In one embodiment, the formulation may comprise a chimeric polynucleotide in linear and circular form. In another embodiment, the formulation may comprise a circular polynucleotide and an IVT polynucleotide. In yet another embodiment, the formulation may comprise an IVT polynucleotide, a chimeric polynucleotide and a circular polynucleotide.

### V. Liposomes, Lipoplexes, and Lipid Nanoparticles

The NAVs of the invention can be formulated using one or more liposomes, lipoplexes, or lipid nanoparticles (LNP). In one embodiment, pharmaceutical compositions of NAVs include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

Peptides and Proteins. The NAVs of the invention can be formulated with peptides and/or proteins in order to increase transfection of cells by the polynucleotide. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3 (3): 310-8 (2001); Langel, Cell-Penetrating Peptides: Processes and Applications (CRC Press, Boca Raton Fla., 2002); El-Andaloussi et al., Curr. Pharm. Des. 1 (28): 3597-611 (2003); and Deshayes et al., Cell. Mol. Life Sci. 62 (16): 1839-49 (2005), all of which are incorporated herein by reference in their entirety). The compositions can also be formulated to include a cell penetrating agent, e.g., liposomes, which enhance delivery of the compositions to the intracellular space. NAVs of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in its entirety).

Cells. The NAVs of the invention can be transfected ex vivo into cells, which are subsequently transplanted into a subject. As one non-limiting example, a sample of blood from a patient or subject may be treated with an antigen or adjuvant or both where one or more are encoded by the NAVs of the invention to activate the PBMC population. This activated sample or a subset of specific cells may then be given back to the donor patient thereby activating the immune system. This activated PBMC vaccine may be designed using any of the NAVs of the present disclosure. As non-limiting examples, the pharmaceutical compositions may include red blood cells to deliver modified RNA to liver and myeloid cells, virosomes to deliver modified RNA in virus-like particles (VLPs), and electroporated cells such as, but not limited to, from MAXCYTE® (Gaithersburg, Md.) and from ERYTECH® (Lyon, France) to deliver modified RNA. Examples of use of red blood cells, viral particles and electroporated cells to deliver payloads other than polynucleotides have been documented (Godfrin et al., Expert Opin Biol Ther. 2012 12:127-133; Fang et al., Expert Opin Biol Ther. 2012 12:385-389; Hu et al., Proc Nat Acad Sci USA. 2011 108:10980-10985; Lund et al., Pharm Res. 2010 27:400-420; Huckriede et al., J Liposome Res. 2007; 17:39-47; Cusi, Hum Vaccin. 2006 2:1-7; de Jonge et al., Gene Ther. 2006 13:400-411; all of which are herein incorporated by reference in its entirety).

Suspension Formulations. In some embodiments, suspension formulations are provided comprising NAVs, water immiscible oil depots, surfactants and/or co-surfactants and/or co-solvents. Combinations of oils and surfactants may enable suspension formulation with NAVs. Delivery of NAVs in a water immiscible depot may be used to improve bioavailability through sustained release of NAVs from the depot to the surrounding physiologic environment and prevent polynucleotides degradation by nucleases.

In some embodiments, suspension formulations of NAV may be prepared using combinations of polynucleotides, oil-based solutions and surfactants. Such formulations may be prepared as a two-part system comprising an aqueous phase comprising polynucleotides and an oil-based phase comprising oil and surfactants. Exemplary oils for suspension formulations may include, but are not limited to sesame oil and Miglyol (comprising esters of saturated coconut and palmkernel oil-derived caprylic and capric fatty acids and glycerin or propylene glycol), corn oil, soybean oil, peanut oil, beeswax and/or palm seed oil. Exemplary surfactants may include, but are not limited to Cremophor, polysorbate 20, polysorbate 80, polyethylene glycol, transcutol, Capmul®, labrasol, isopropyl myristate, and/or Span 80. In some embodiments, suspensions may comprise co-solvents including, but not limited to ethanol, glycerol and/or propylene glycol.

Cryoprotectants. In some embodiments, NAV formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with NAVs in order to stabilize them during freezing. Frozen storage of NAVs between −20° C. and −80° C. may be advantageous for long term (e.g. 36 months) stability of polynucleotide. In some embodiments, cryoprotectants are included in NAV formulations to stabilize polynucleotide through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

Bulking Agents. In some embodiments, NAV formulations may comprise bulking agents. As used herein, there term “bulking agent” refers to one or more agents included in formulations to impart a desired consistency to the formulation and/or stabilization of formulation components. In some embodiments, bulking agents are included in lyophilized NAV formulations to yield a “pharmaceutically elegant” cake, stabilizing the lyophilized NAVs during long term (e.g. 36 month) storage. Bulking agents of the present invention may include, but are not limited to sucrose, trehalose, mannitol, glycine, lactose and/or raffinose. In some embodiments, combinations of cryoprotectants and bulking agents (for example, sucrose/glycine or trehalose/mannitol) may be included to both stabilize NAVs during freezing and provide a bulking agent for lyophilization.

Administration. The NAVs of the present invention may be administered by any route which results in a therapeutically effective outcome. Liquid dosage forms for parenteral administration include, but are not limited to, pharmaceutically acceptable emulsions, microemulsions, solutions, suspensions, syrups, and/or elixirs.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Vaccination or immunization can be performed using a vaccination or immunization regimen; for instance, administering one or more vaccines or immunological or immunogenic compositions as a “prime” and thereafter administering one or more vaccines or immunological or immunogenic compositions as a “boost”. The prime-boost regimen according to the invention can be used in subjects of any age. The term of “prime-boost” refers to the successive administrations of two vaccines or immunogenic or immunological compositions having at least one immunogen, antigen or epitope in common. The priming administration (priming) is the administration of a first vaccine or immunogenic or immunological composition type and may comprise one, two or more administrations. The boost administration is the administration of a second vaccine or immunogenic or immunological composition type and may comprise one, two or more administrations, and, for instance, may comprise or consist essentially of annual administrations. The prime and boost can be the same or different compositions as long as there is a common immunogen, antigen, or epitope presented to the subject.

Dosing. The present invention provides methods comprising administering NAVs and in accordance with the invention to a subject in need thereof. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

According to the present invention, NAVs may be administered in split-dose regimens. As used herein, a “split dose” is the division of single unit dose or total daily dose into two or more doses, e.g., two or more administrations of the single unit dose. As used herein, a “single unit dose” is a dose of any therapeutic administer in one dose/at one time/single route/single point of contact, i.e., single administration event. As used herein, a “total daily dose” is an amount given or prescribed in 24 hr period. It may be administered as a single unit dose. In one embodiment, the NAVs of the present invention are administer to a subject in split doses. The NAVs may be formulated in buffer only or in a formulation described herein.

In some embodiments, NAV compounds and/or compositions of the present invention may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some embodiments, multi-dose administration may comprise repeat-dose administration. As used herein, the term “repeat-dose administration” refers to two or more doses administered consecutively or within a regimen of repeat doses comprising substantially the same components provided at substantially the same mass and/or volume. In some embodiments, subjects may display a repeat-dose response. As used herein, the term “repeat-dose response” refers to a response in a subject to a repeat-dose that differs from that of another dose administered within a repeat-dose administration regimen. In some embodiments, such a response may be the expression of a protein in response to a repeat-dose comprising NAV. In such embodiments, protein expression may be elevated in comparison to another dose administered within a repeat-dose administration regimen or protein expression may be reduced in comparison to another dose administered within a repeat-dose administration regimen. Alteration of protein expression may be from about 1% to about 20%, from about 5% to about 50% from about 10% to about 60%, from about 25% to about 75%, from about 40% to about 100% and/or at least 100%. A reduction in expression of mRNA administered as part of a repeat-dose regimen, wherein the level of protein translated from the administered RNA is reduced by more than 40% in comparison to another dose within the repeat-dose regimen is referred to herein as “repeat-dose resistance.”

### VI. Kits and Devices

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In one aspect, the present invention provides kits comprising the NAV molecules (including any proteins or polynucleotides) of the invention. In one embodiment, the kit comprises one or more functional antigens or function fragments thereof.

The kits can be for protein production, comprising a first polynucleotides comprising a translatable region of an antigen. The kit may further comprise packaging and instructions and/or a delivery agent to form a formulation composition. The delivery agent may comprise a saline, a buffered solution, or a delivery agent.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose. In a further embodiment, the buffer solutions may be precipitated or it may be lyophilized. The amount of each component may be varied to enable consistent, reproducible higher concentration saline or simple buffer formulations. The components may also be varied in order to increase the stability of polynucleotides in the buffer solution over a period of time and/or under a variety of conditions.

In one aspect, the present invention provides kits for protein production, comprising: a polynucleotide comprising a translatable region, provided in an amount effective to produce a desired amount of a protein encoded by the translatable region when introduced into a target cell.

Devices. The present invention provides for devices which may incorporate RNAVs comprising polynucleotides that encode polypeptides of interest, e.g., encode antigenic polypeptides. These devices contain in a stable formulation the reagents to synthesize a polynucleotide in a formulation available to be immediately delivered to a subject in need thereof, such as a human patient.

Devices for administration may be employed to deliver the NAVs of the present invention according to single, multi- or split-dosing regimens taught herein.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

In one embodiment, the NAV is administered subcutaneously or intramuscularly via at least 3 needles to three different, optionally adjacent, sites simultaneously, or within a 60 minutes period (e.g., administration to 4, 5, 6, 7, 8, 9, or 10 sites simultaneously or within a 60 minute period).

### VII. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Methods

Generation of mRNA-N and mRNA-S. Modified mRNA encoding prefusion-stabilized SARS-CoV-2 S protein (mRNA-S-2P; or refer to as mRNA-S) and modified mRNA encoding SARS-CoV-2 N protein (mRNA-N) were synthesized in vitro using T7 RNA polymerase (Pardi et al., Methods Mol Biol, 2013, 969:29-42). One methylpseudouridine (m1Ψ)-5′-triphosphate, instead of UTP was used to produce nucleoside-modified mRNAs. Modified mRNAs were 5′-capped and contain polyadenylation tails for optimized expression. All mRNAs were purified by cellulose purification (Baiersdorfer et al., Mol Ther Nucleic Acids, 2019, 15:26-35).

mRNA-LNP packaging. mRNAs were encapsulated in lipid nanoparticles (LNP) for animal immunization (Maier et al., Mol Ther, 2013, 21 (8): 1570-8). LNPs formulations contain ionizable cationic lipid, phosphatidylcholine, cholesterol, and PEG-lipid.

Mouse immunization, sample collection, and SARS-CoV-2 challenge. An animal study was conducted in accordance with the recommendations in the Guide for the Care and Use of Laboratory Animals of the National Institutes of Health. Animal protocol was approved by the Institutional Animal Care and Use Committee (IACUC) at the University of Texas Medical Branch. For mouse study, 6-week old female BALB/c mice (5 per group) were immunized intramuscularly (i.m.) with either PBS (50 μl) as the mock control or 1 μg mRNA-N vaccine (50 μl) using a prime-boost approach at week 0 (prime) and week 3 (boost), respectively.

Blood/serum samples were collected from all mice one day before boost (post-prime) as well as one day before challenge (post-boost). Two weeks after the 2^{nd} vaccination (week 5), all mice were intranasally challenged with mouse-adapted SARS-CoV2 CMA4 strain (2×10⁴ pfu) (Muruato et al., bioRxiv, 2021; Ku et al., Nat Commun, 2021, 12 (1): 469). 2 days post infection (2 DPI), mice were euthanized and equivalent portions of lung tissues were collected for quantification of viral loads by q-PCR or by plaque assay.

Hamster immunization, sample collection, and SARS-CoV-2 challenge. 5 groups of 3-week old male golden Syrian hamsters (n=12 per group; 6 for 2 DPI and 6 for 4 DPI) respectively received PBS (mock), mRNA-S (2 μg), mRNA-N (2 μg), mRNA-S+N (2 μg for each), mRNA-N (2 μg) (with CD8 depletion), at week 0 (prime) and week 3 (boost), followed by intranasal challenge with SARS-CoV-2 delta variant (10⁴ pfu). Prior to challenge, blood/serum samples were collected from each hamster one-day before boost (post-prime) and one-day before challenge (post-boost) for serological analysis. 2 days and 4 days post infection (2 DPI and 4 DPI), all hamsters were euthanized and nasal wash samples as well as equivalent portions of lung tissues were collected for quantification of viral loads by q-PCR or by plaque assay.

Binding IgG by ELISA. ELISA was used to measure N-specific binding IgG in mouse sera. ELISA plates (Greiner bio-one) were coated with 1 μg/ml recombinant N protein (40588-V08B; Sino Biological) in DPBS overnight at 4° C. Plates were washed three times with wash buffer (DPBS with 0.05% Tween 20), 5 min for each time, and then blocked with 8% FBS in DPBS for 1.5 hour at 37° C. Plates were washed and incubated with serially diluted sera in blocking buffer at 50 μl per well for 1 hour at 37° C. For quantification of binding antibodies in BAL, collected BAL fluids were used for incubation without dilution. ELISA was conducted in duplicate. Plates were again washed and incubated with horse radish peroxidase (HRP) conjugated anti-mouse IgG secondary antibody (Biolegend) (1:5000) for 1 hour at 37° C. After final wash, plates were developed using TMB 1-Component Peroxidase Substrate (Thermo Fisher), followed by termination of reaction using the TMB stop solution (Thermo Fisher). Plates were read at 450 nm wavelength within 30 min by using a Microplate Reader (BioTek).

RT-PCR quantification of viral copies in lung tissue. Lung tissues were weighted and homogenized, followed by RNA extraction using Trizol LS (Invitrogen). Extracted RNA quality and quantify were examined by microplate reader (BioTek). SARS-CoV-2 viral copies were quantified by one-step quantitative reverse transcription PCR (RT-qPCR) (Bio-Rad) using primers specific for viral E gene. Pure viral E RNA with known quantity was used for generation of standard curve. Absolute viral copy numbers were quantified using the standard curve and were normalized to lung tissue weights. Data were shown as Log 10 viral copies/mg lung tissue.

### Example 2

### Combination mRNA Vaccination Induces Robust Protection Against SARS-CoV-2 Omicron and Delta Variants

### A. Results

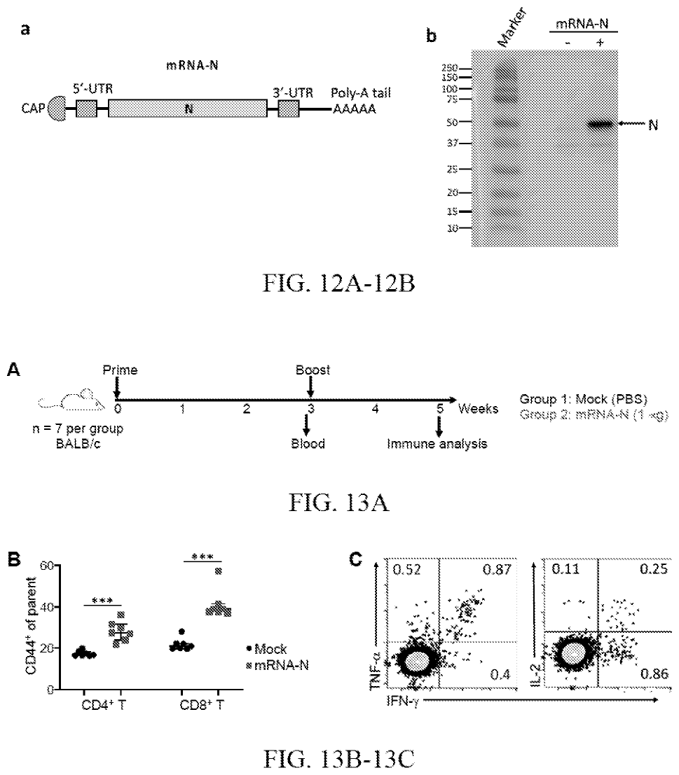

mRNA-N vaccine generation and immunogenicity analysis. Since the coronavirus N protein represents an important viral antigen to induce durable and broadly reactive T cells, a methyl-psuedouridine-modified (m1Ψ) mRNA was designed and generated that encodes the full-length N protein of SARS-CoV-2 (Wuhan-Hu-1 strain) (FIG. 12A). Synthesis, purification, and lipid nanoparticle (LNP) formation of the mRNA-N vaccine were conducted as previously described (Pardi et al., Methods Mol Biol 969, 29-42, 2013; Baiersdorfer et al., Mol Ther Nucleic Acids 15, 26-35, 2019; Maier et al., Mol Ther 21, 1570-78, 2013). Expression of the N protein in cells following mRNA-N transfection was confirmed by western blot (FIG. 12B).

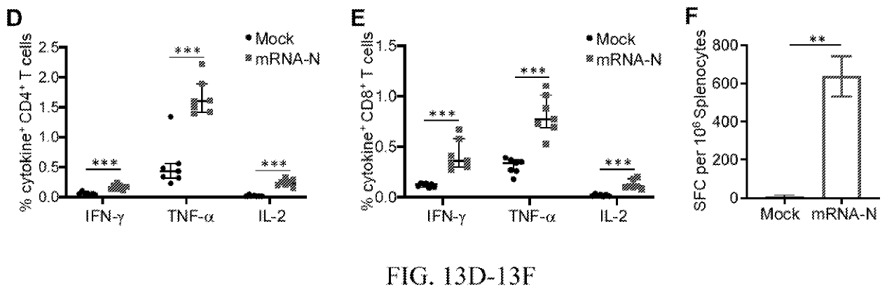

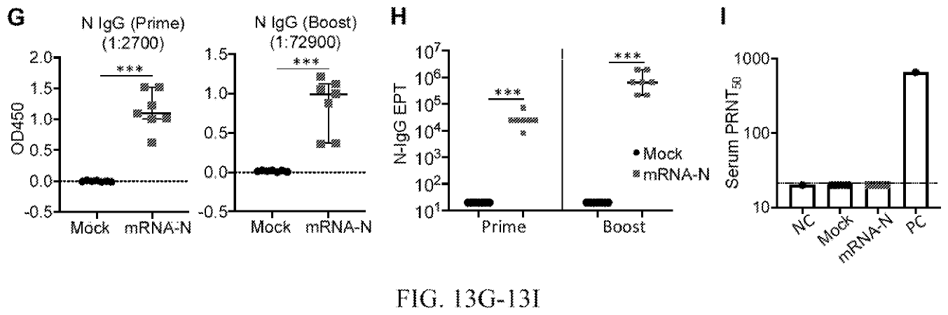

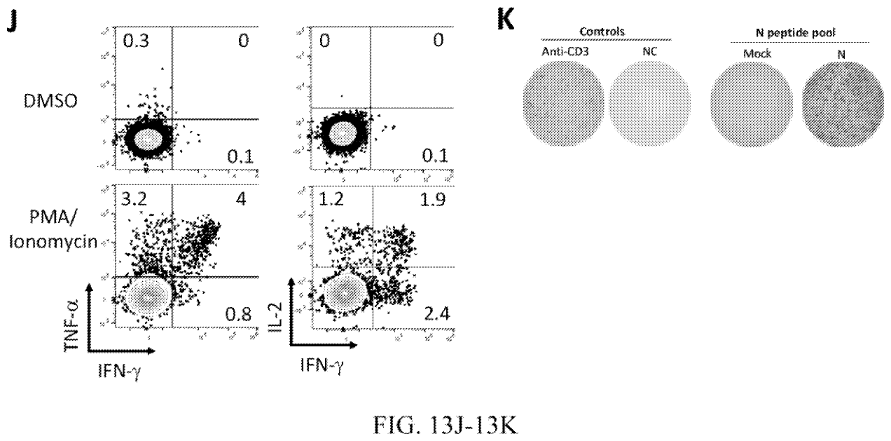

The immunogenicity of mRNA-N was evaluated in BALB/c mice (FIG. 13A). Two groups of mice (n=7 per group) were vaccinated with phosphate-buffered saline (PBS, mock) or mRNA-N (1 μg), a dose selected based on previous studies testing similar mRNA-LNP in mice (Corbett et al., Nature 586, 567-71, 2020). Vaccination was given intramuscularly (i.m.) at week 0 (prime) and week 3 (boost). Three weeks after prime vaccination (on the day of the booster), serum samples were collected for analysis of antibody responses; two weeks after the booster (week 5), mice were euthanized and subjected to immunological analyses (FIG. 13A). First, T cell response was examined in splenocytes by flow cytometry. Based on CD44 expression, a marker used for T cell activation and memory (Ponta et al., Nat Rev Mol Cell Biol 4, 33-45, 2003; Baaten and Bradley, Commun Integr Biol 3, 508-12, 2010), it was observed that, compared to the mock controls, mRNA-N induced strong CD4⁺ and CD8⁺ T cell activation in the spleen (FIG. 13B). The N-specific T cell response was measured by intracellular cytokine staining (ICS), following stimulation of splenocytes with a 15-mer peptide pool that spans the entire N protein (QHD43423.2). Representative flow cytometry plots for cytokine expression in T cells are shown in FIG. 13C and FIG. 13J. Compared to the mock controls, mRNA-N induced high N-specific CD4⁺ and CD8⁺ T cell responses (p<0.001 for all three cytokines) (FIGS. 13D and 13E). N-specific T cells appeared to predominantly express tumor necrosis factor (TNF)-α (median: 1.60% for CD4⁺ T cells and 0.77% for CD8⁺ T cells), followed by interferon (IFN)-γ and interleukin (IL)-2 (FIGS. 13D and 13E). The mRNA-N vaccine-induced T cell response was also evaluated by an IFN-γ enzyme-linked immunosorbent spot (ELISPOT) assay (FIG. 13K). Compared to mock controls, the vaccine elicited high numbers of N-specific T cells in the spleen (median spot-forming cells (SFC)/10⁶ splenocytes for mock versus mRNA-N: 6 versus 638) (p<0.01) (FIG. 13F).

Antibodies in the mouse serum samples were examined following prime and booster immunization. Compared to the mock controls, prime immunization induced strong N-specific binding IgG responses (FIG. 13G; left panel), which were further enhanced by the booster (FIG. 13G; right panel). To determine antibody endpoint titers (EPTs), serum samples were serially diluted and N-specific binding IgG was examined by ELISA (FIG. 13L). The analysis showed that median IgG EPTs after prime and booster vaccination were 24,300 and 656, 100, respectively (FIG. 13H). Finally, serum neutralizing activity was determined by the Plaque Reduction Neutralization Test (PRNT). As expected, based on the lack of exposure to the S protein that mediates viral entry, no neutralizing activity was detected in any of the vaccinated animals (FIG. 13I). Together, these data suggest that the mRNA-N vaccine is highly immunogenic and i.m. immunization induces a robust N-specific T cell immunity and binding antibody response.

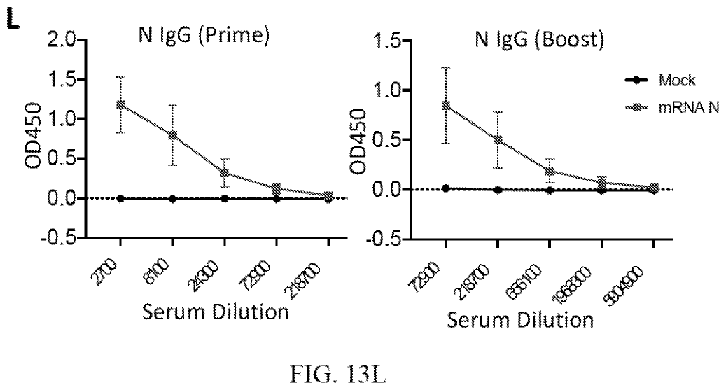

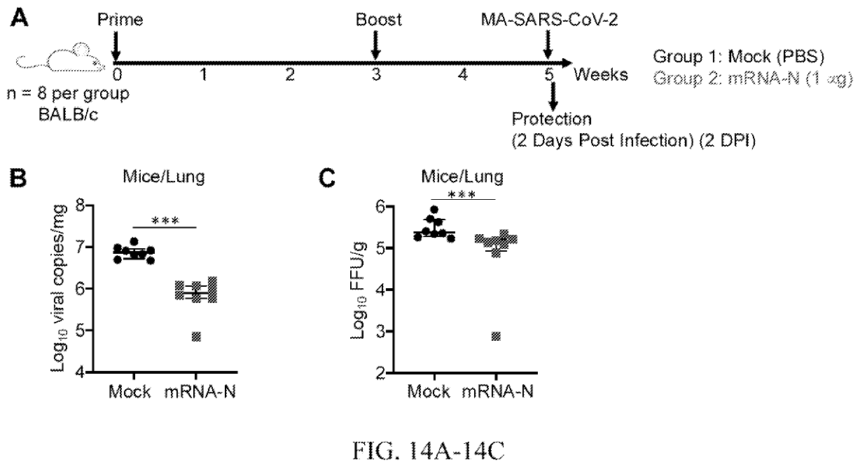

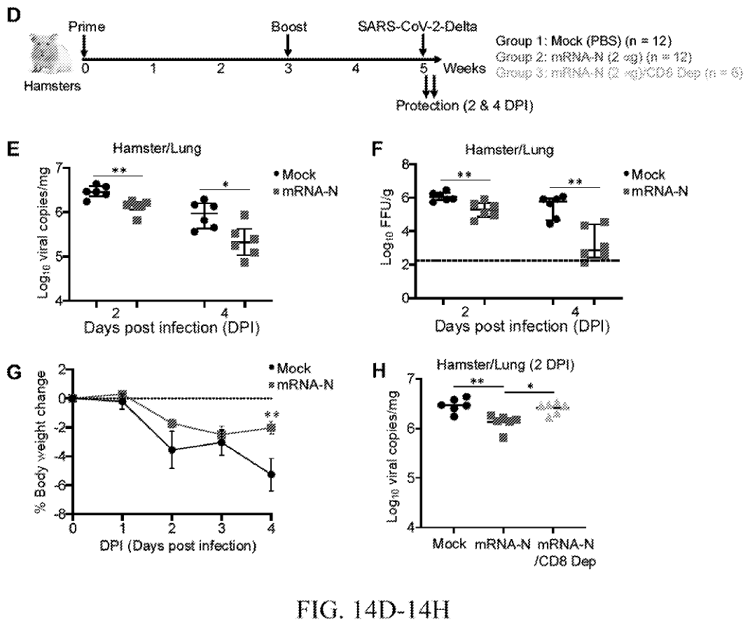

mRNA-N alone induces modest but significant control of SARS-CoV-2 in mice and hamsters. It remained unclear if immunization with the N-expressing vaccine alone would induce immune-mediated control of SARS-CoV-2 infection. The effectiveness of mRNA-N in animal models was evaluated. First, two groups of BALB/c mice (n=8 per group) were vaccinated with either PBS (mock) or mRNA-N vaccine at week 0 (prime) and week 3 (boost), followed by intranasal challenge with a mouse-adapted SARS-CoV-2 strain (MA-SARS-CoV-2; 2×10⁴ plaque forming units [pfu]) (Ku et al., Nat Commun 12, 469, 2021) at week 5 (FIG. 14A). Two days post-infection (DPI), viral loads in the lungs were quantified for viral RNA and infectious titers. Compared to the mock-immunized controls, i.m. immunization with mRNA-N induced a modest but significant reduction in viral RNA copies (p<0.001) (FIG. 14B), as well as in viral titers (p<0.001) (FIG. 14C). The vaccine's protective effect in mice following intranasal (i.n.) immunization using the same vaccination schedule and challenge dose as in the i.m. route was also evaluated. In contrast to i.m. vaccination, i.n. vaccination did not induce notable viral control in the lungs (FIGS. 141 and 14J). Consistent with the lack of protection, no antibody response (binding IgG) was induced in serum samples following mRNA-N i.n. immunization (FIG. 14K). Thus, i.m. immunization was employed in all subsequent experiments.

Next, the vaccine against the SARS-CoV-2 Delta variant was evaluated in hamsters, which are susceptible to wild-type SARS-CoV-2 strains. Three cohorts were investigated (FIG. 14D). The first two (n=12 per group) were i.m. vaccinated with PBS (mock) or the mRNA-N vaccine (2 μg) at week 0 and 3, followed by i.n. challenge with the SARS-CoV-2 Delta strain (2×10⁴ pfu). A higher vaccine dose (2 μg) was selected based on previous studies evaluating similar mRNA-LNP COVID-19 vaccines in hamsters (Imai et al., Proc Natl Acad Sci USA 117, 16587-95, 2020; Meyer et al, J Clin Invest 131, 2021). For each group, on 2 DPI (n=6) and 4 DPI (n=6), viral RNA copies and infectious titers in the lungs were assayed, along with viral RNA copies in nasal washes. In addition, a third group (n=6) that received the mRNA-N vaccine but underwent in vivo CD8⁺ T cell depletion was included (FIG. 14D). These hamsters received two doses of CD8-depletion antibody (Hammerbeck and Hooper, J Virol 85, 9929-44, 2011; Prescott et al., Immunology 140, 168-178, 2013) at 6 and 3 days prior to challenge and viral RNA copies in the lungs were analyzed on 2 DPI (n=6). The data showed that on 2 DPI, compared to the mock control, mRNA-N induced a statistically significant but modest impact on viral RNA copies and infectious titers (FIGS. 14E and 14F) (p<0.01). On 4 DPI, compared to the mock controls, a more profound effect was observed for the vaccine in reducing viral RNA copies (3-fold reduction; p<0.05) (FIG. 14E) and infectious titers (173-fold reduction; p<0.01) (FIG. 14F).

Hamster body weights were monitored on the day of challenge through 4 DPI. Infection with Delta led to considerable weight loss in the mock-vaccinated group (greater than 5% on 4 DPI); mRNA-N-vaccinated hamsters exhibited reduced weight loss at 4 DPI as compared to mock-vaccinated hamsters (p<0.05) (FIG. 14G). These data indicated modest protection conferred by mRNA-N vaccine against SARS-CoV-2 Delta challenge. In addition, we compared viral RNA copies in the lungs of hamsters that received mRNA-N vaccine with or without CD8 T cell depletion (FIG. 14H). CD8 T cell depletion largely abrogated the effect of the vaccine on viral control (FIG. 14H), indicating a potential role for CD8 T cells in mRNA-N-induced protection against SARS-CoV-2 Delta challenge. Finally, compared to the mock controls, mRNA-N vaccination did not reduce viral loads in the nasal washes (FIG. 14L). These results indicate that mRNA-N alone has minimal impact in the upper respiratory tract, likely due to the vaccine's inability to induce neutralizing antibodies.

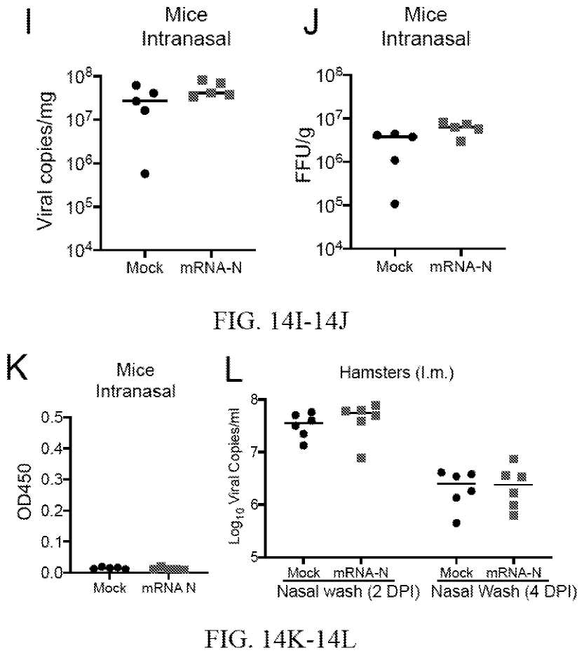

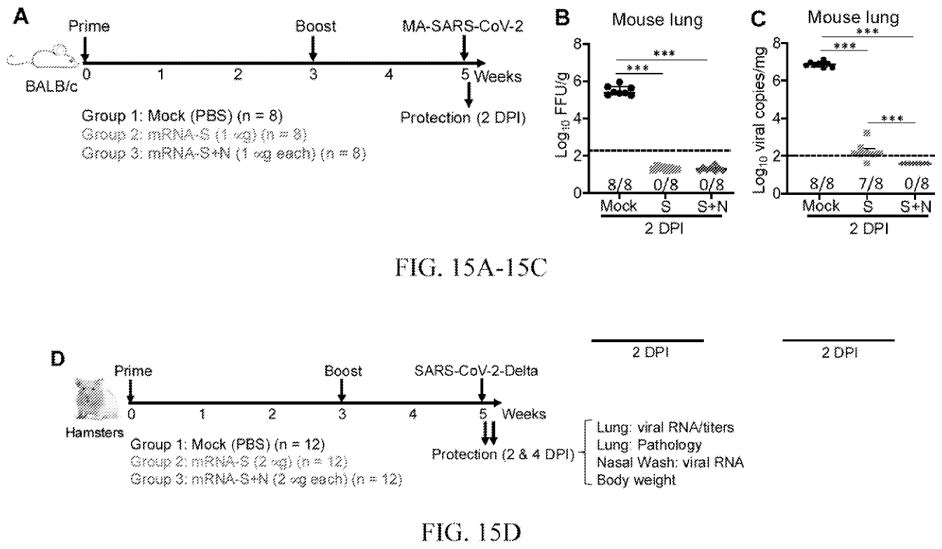

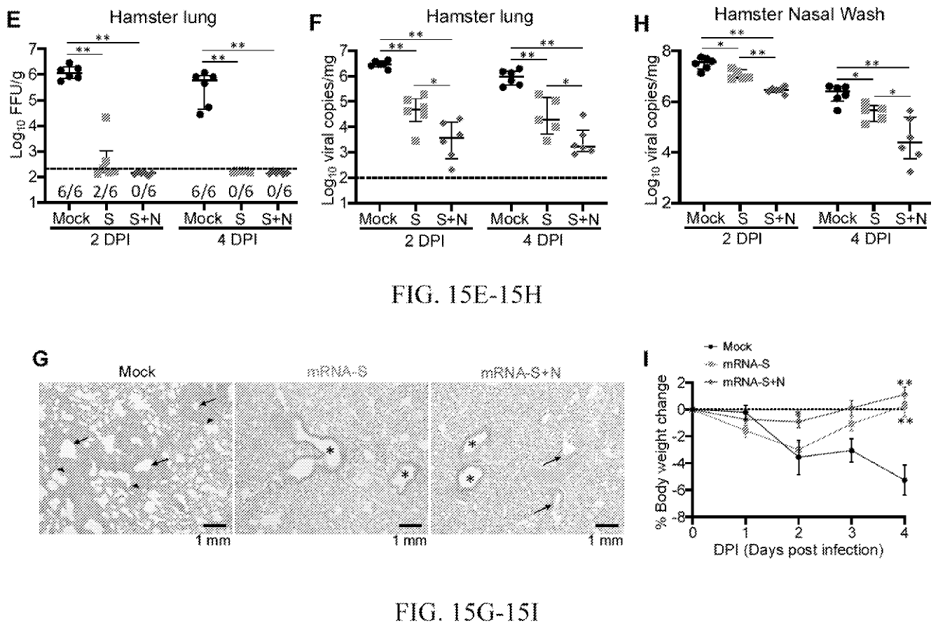

Combinatorial mRNA vaccination induces robust protection against SARS-CoV-2 Delta variant. After demonstrating that mRNA-N alone was immunogenic and elicited modest efficacy against SARS-CoV-2, a bivalent vaccine consisting of both mRNA-N and the S-expressing mRNA vaccine (mRNA-S) was tested for inducing a more robust protection against variants. Thus, in addition to mRNA-N, an m1Ψ-modified mRNA vaccine was also generated that expresses the prefusion-stabilized SARS-CoV-2 S protein with two proline mutations (named “mRNA-S-2P”) (Wuhan-Hul), similar to Pfizer/BioNTech's BNT162b (Polack et al., N Engl J Med 383, 2603-15, 2020) and Moderna's mRNA-1273 (Baden et al., N Engl J Med 384, 403-16, 2021) vaccines. First, these vaccines were tested in BALB/c mice. Three groups of mice (n=8 per group) were immunized with either PBS (mock), mRNA-S (1 μg), or the combination mRNA-S/mRNA-N vaccine (mRNA-S+N; 1 μg of each mRNA) at weeks 0 and 3, followed by i.n. challenge with the MA-SARS-CoV-2 strain (2×10⁴ pfu) at week 5; on 2 DPI, viral titers and RNA copies in the lungs were measured (FIG. 15A). Compared to mock vaccination, both mRNA-S alone and the combined mRNA-S+N induced complete viral control with no detectable infectious virus in the lungs (FIG. 15B). However, quantification of viral RNA by the more-sensitive RT-PCR approach revealed a significant difference between mRNA-S and mRNA-S+N (p<0.001) (FIG. 15C). Compared to the mock control, mRNA-S alone remained highly effective in reducing viral RNA copies in the lungs (7 of 8 with weakly detectable RNA and 1 of 8 with no detectable RNA); however, mRNA-S+N induced complete protection against viral RNA in the lungs in all eight mice tested (FIG. 15C).

Second, these vaccines were tested against the Delta variant in hamsters. Three groups (n=12 per group) were vaccinated with PBS (mock), mRNA-S (2 μg), or mRNA-S+N (2 μg for each) at weeks 0 and 3, followed by i.n. challenge at week 5. On 2 (n=6) and 4 DPI (n=6), hamsters were analyzed for vaccine-induced protection based on viral loads, lung histopathologic lesions, and body weight loss (FIG. 15D). On 2 DPI, compared to mock vaccinations, mRNA-S alone induced substantial control of infectious virus in the lungs (2 of 6 with detectable titers), whereas mRNA-S+N induced complete viral control with no detectable titers in any of the 6 hamsters (FIG. 15E). On 4 DPI, both mRNA-S and mRNA-S+N vaccinations completely controlled virus in the lungs (FIG. 15E). Analysis of viral RNA revealed a more profound difference between mRNA-S and mRNA-S+N. Compared to mock-vaccination, mRNA-S alone reduced lung viral RNA copies by 57-fold (p<0.01) (FIG. 15F). Critically, relative to mRNA-S, mRNA-S+N induced a more robust viral control on 2 DPI and reduced viral RNA copies by an additional 12-fold (p<0.05 for mRNA-S versus mRNA-S+N) (FIG. 15F). Compared to mock-vaccination, mRNA-S+N induced a 770-fold reduction in median viral RNA copies (FIG. 15F). A similar result was observed on 4 DPI (FIG. 15F). Consistent with the viral load data, lung histopathological analysis showed that on 2 DPI, Delta challenge caused evident changes in the mock-immunized hamsters, including bronchiolitis and interstitial pneumonia (FIG. 15G). Hamsters vaccinated with mRNA-S or mRNA-S+N were all protected from these lesions and demonstrated normal bronchial, bronchiolar, and alveolar architecture (FIG. 15G). The data indicate that mRNA-S vaccine itself is effective against disease caused by SARS-CoV-2 Delta, consistent with clinical findings (Lopez Bernal et al., N Engl J Med 385, 585-94, 2021), and further show that mRNA-S+N was highly protective in both models.

Viral RNA copies in nasal washes were examined on 2 DPI and 4 DPI. Unlike the robust viral control provided by mRNA-S in the lungs, mRNA-S was less effective in reducing viral RNA copies in the nasal washes on both 2 (mock versus mRNA-S: 3-fold reduction) and 4 DPI (5-fold reduction) (FIG. 15H). These data suggest that mRNA-S induces strong protection against disease but reduced protection against infection and upper airway shedding by the Delta variant (Tang et al., Nat Med, 2021). Compared to mRNA-S alone, mRNA-S+N induced more robust viral control in the nasal washes on 2 DPI (mock versus mRNA-S+N: 11-fold reduction; p<0.01) and 4 DPI (98-fold reduction; p<0.05) (FIG. 15H). Together, these data support that combination mRNA-S+N vaccination induces stronger and faster control of SARS-CoV-2 Delta infection in both lungs and the upper respiratory tract as compared to mRNA-S alone, indicating that this vaccine approach may also reduce the risk of transmission.

Body weights analysis showed that challenge with the Delta variant caused progressive weight loss in the mock-vaccinated hamsters, declining by greater than 5% on 4 DPI (FIG. 15I), which is comparable with that caused by the wild-type SARS-CoV-2 (Plante et al., Nature 592, 116-21, 2021). Compared to mock vaccination, mRNA-S alone or the bivalent mRNA-S+N protected hamsters from weight loss on 3 DPI and 4 DPI (FIG. 15I). Finally, compared to mRNA-S alone, mRNA-S+N resulted in significantly reduced weight loss at 2 DPI (p<0.05) (FIG. 15I).

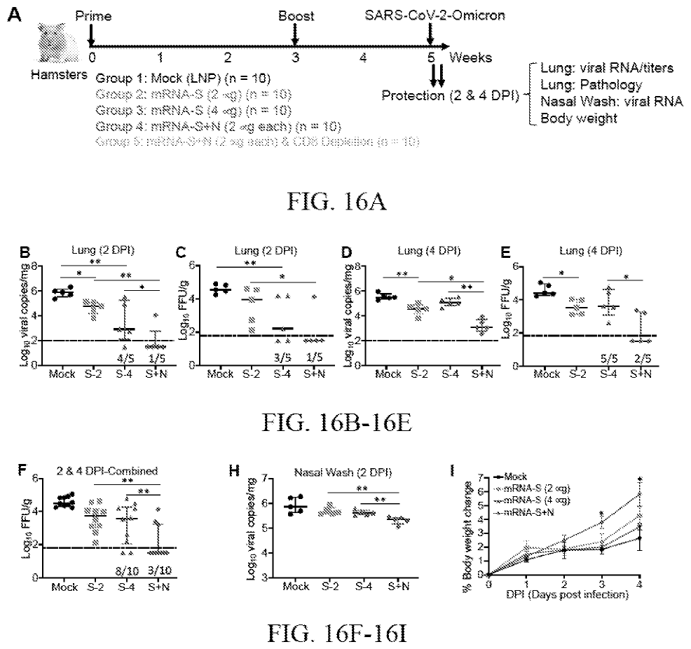

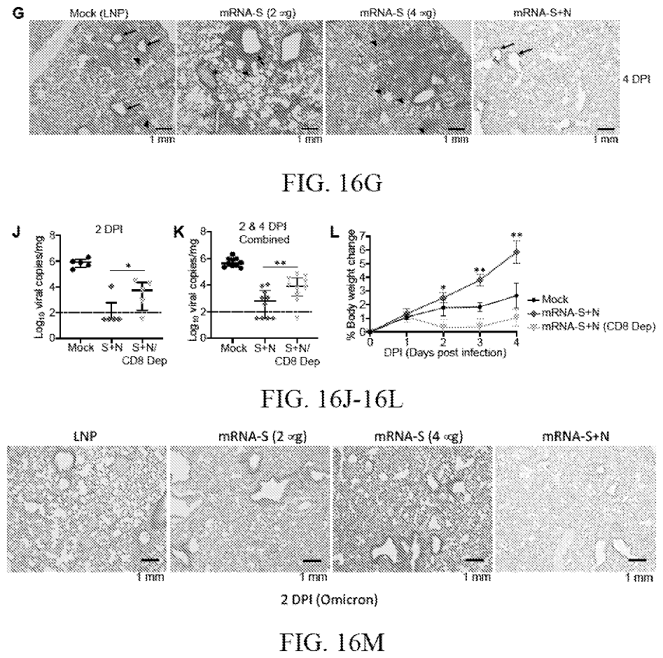

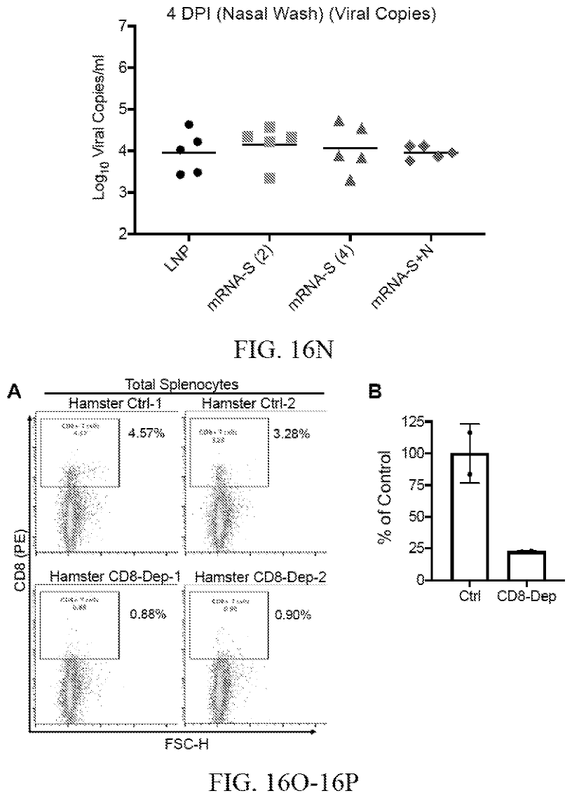

Combinatorial mRNA vaccination induces robust protection against SARS-CoV-2 Omicron variant. The efficacy of mRNA-S+N vaccination against the SARS-CoV-2 Omicron variant (BA.1) was investigated. First, 4 groups of hamsters (n=10) were vaccinated with empty LNP (mock), mRNA-S (2 μg), mRNA-S (4 μg), or mRNA-S+N (2 μg for each mRNA) at weeks 0 and 3 (FIG. 16A). The mRNA-S (4 μg) dose group was included as another control to determine if enhanced protection by mRNA-S+N was due to the higher total dose of mRNA or LNP. Two weeks after the booster (week 5), all hamsters were intranasally challenged with the SARS-CoV-2 Omicron strain (2×10⁴ pfu). On 2 (n=5) and 4 DPI (n=5), protection was analyzed based on viral loads, histopathologic changes in the lungs, and body weight changes (FIG. 16A). On 2 DPI, compared to the empty-LNP control, mRNA-S (2 μg) induced only modest control of the Omicron variant in the lungs based on viral RNA copies (12-fold reduction) and infectious titers (3-fold reduction) (FIGS. 16B and 16C). Compared to 2 μg mRNA-S, vaccination with 4 μg mRNA-S induced comparable viral control on both 2 and 4 DPI, based on viral RNA copies (FIGS. 16B and 16D) and infectious titers (FIGS. 16C and 16E). These data indicate that a higher mRNA-S dose did not provide markedly stronger protection against the Omicron. Critically, compared to mRNA-S alone (either 2 μg or 4 μg), combination mRNA-S+N induced more robust control of Omicron based on viral RNA copies (FIGS. 16B and 16D) and infectious titers (FIGS. 16C and 16E). On 2 DPI, mRNA-S+N induced complete viral control with no detectable viral RNA in 4 out of 5 hamsters (FIG. 16B). Viral titers yielded comparable results: no detectable infectious virus in 4 out of 5 hamsters in the mRNA-S+N group, whereas 4 out of 5 hamsters in the mRNA-S (4 μg) group had detectable virus (FIG. 16C). A similar result was observed on 4 DPI. Relative to mRNA-S alone, mRNA-S+N vaccination further reduced median viral RNA copies by 100-fold (FIG. 16D). Finally, pooled analysis of viral titers for 2 and 4 DPI lung samples revealed a significant difference between mRNA-S and mRNA-S+N groups (p<0.01) (FIG. 16F).

Histopathological analysis showed no changes in the lungs in any hamsters on 2 DPI, including the mock-vaccinated controls (FIG. 16M). However, considerable changes, including bronchitis and interstitial pneumonia, became evident on 4 DPI (FIG. 16G). Compared to the mock-vaccinated controls, hamsters vaccinated with mRNA-S alone, either at the 2 or 4 μg dose, still developed lesions including interstitial pneumonia and peribronchitis; critically, mRNA-S+N vaccination fully protected hamsters from all lesions with normal bronchial, bronchiolar, and alveolar architecture (FIG. 16G). This finding is consistent with the strong protection indicated by the above viral load data.

Analysis of viral RNA in the nasal washes indicated that mRNA-S vaccination (2 or 4 μg) had limited effects on SARS-CoV-2 Omicron shedding in the upper respiratory tract and only weakly reduced viral copies compared to the LNP controls (FIG. 16H). This likely indicates a strong immune escape of Omicron from mRNA-S-induced neutralization (Cao et al., Nature 602, 657-63, 2022; Planas et al., Nature 602, 671-75, 2022; Liu et al., Nature 602, 676-81, 2022; Cele et al., Nature 602, 654-56, 2022). This weak protective effect in the nasal washes was further diminished by 4 DPI (FIG. 16N). However, on 2 DPI, compared to mRNA-S alone, mRNA-S+N vaccination induced a significant reduction in viral RNA copies in the nasal washes (3.3-fold reduction; p<0.01) (FIG. 16H), indicating that mRNA-S+N vaccination also provides additional control of Omicron in the upper respiratory tract.

Body weight analysis highlighted differences between Delta and Omicron infection in the hamsters (using the same challenge doses). Infection with the Delta variant produced a progressive decline in body weights (FIG. 15I), whereas hamsters infected with Omicron maintained a steady increase in weights through 4 DPI (FIG. 16I), consistent with reports that Omicron infection is more attenuated in animal models (Halfmann et al., Nature, 2022; Shuai et al., Nature, 2022; Suzuki et al., Nature, 2022). Compared to mRNA-S alone (2 and 4 μg), mRNA-S+N vaccination led to a significant increase in weights (p<0.05 for 3 and 4 DPI) (FIG. 16I). Body weight increases for the mRNA-S+N-vaccinated hamsters were comparable to those of uninfected hamsters reported previously (approximately 6% increase by 4 DPI) (Plante et al., Nature 592, 116-21, 2021). This indicates that mRNA-S+N vaccination protects hamsters from Omicron-induced morbidity, consistent with its robust protection against viral loads and pathologic effects in the lungs.

To explore the potential involvement of CD8 T cells in mRNA-S+N-induced protection, another group of hamsters (n=10) that received the same mRNA-S+N vaccine at weeks 0 and 3 was included. Six and three days prior to the Omicron challenge, hamsters were injected (i.p.) with two doses of antibody for in vivo CD8 T cell depletion (35, 36) (FIG. 16A). CD8 T cell depletion efficiency was confirmed by flow cytometry (FIG. 16O-16P). Compared to mRNA-S+N vaccination without depletion, CD8 T cell depletion resulted in a modest but significant increase in viral copies in the lungs on 2 DPI (p<0.05) (FIG. 16J). Pooled analysis of 2 DPI and 4 DPI samples indicated a significant effect of CD8 T cell depletion on viral RNA copies in the lungs (p<0.01) (FIG. 16K). Body weight analysis showed that, compared to mRNA-S+N or LNP-vaccinated hamsters, CD8 T cell-depleted hamsters had reduced body weight gain on both 2 and 4 DPI (FIG. 16L), also indicating the potential involvement of CD8 T cells in immune protection against Omicron by mRNA-S+N vaccination.

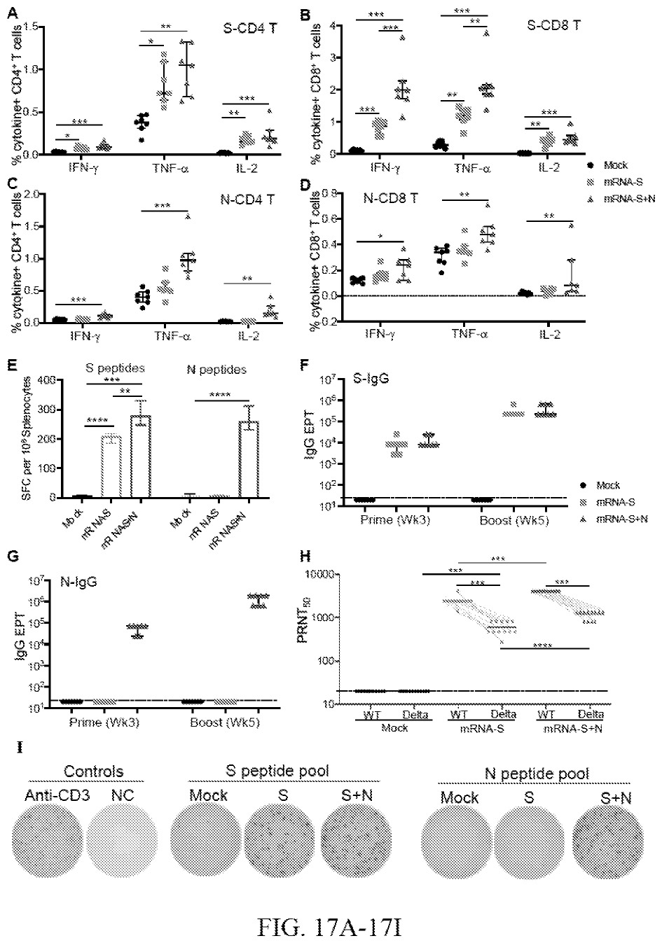

Combinatorial mRNA vaccination elicits robust N- and S-specific T-cell and humoral immunity. To better understand antigen-specific immune responses induced by vaccination, a mouse immunogenicity experiment was conducted, where 3 groups of BALB/c mice (n=7 per group) were vaccinated with PBS (mock), mRNA-S alone, or mRNA-S+N at weeks 0 and 3 using a similar experimental design to that described in FIG. 13A. Mouse splenocytes collected at week 5 were stimulated with S or N peptide pools and ICS was performed to identify S- and N-specific T cells (FIG. 17A-17D). The data showed that combination mRNA-S+N vaccination elicited robust S-specific (FIG. 17A-17B) and N-specific (FIGS. 17C and 17D) CD4⁺ and CD8⁺ T cell responses. Among the cytokines examined, TNF-α was highly expressed by both S- and N-specific T cells, followed by IFN-γ and IL-2 (FIG. 17A-17D). Compared to mRNA-S alone, the mRNA-S+N vaccination appeared to augment the S-specific CD8⁺ T cell response (p<0.001 for IFN-γ⁺, p<0.01 for TNF-α⁺) (FIG. 17B). Induction of S- and N-specific T cells by mRNA-S+N vaccination compared to mRNA-S alone was also confirmed by IFN-γ ELISPOT (FIG. 17E, 17I).

Serum binding IgG to S or N was measured by ELISA and the data revealed similar patterns (FIG. 17F, 17G). The mRNA-S alone induced robust binding IgG targeted to S (FIG. 17F), but not to N (FIG. 17G), following prime vaccination, which was markedly enhanced by the booster vaccination (FIG. 17F). Compared to mRNA-S alone, combination mRNA-S+N elicited strong binding IgG specific to both S and N proteins following prime vaccination, both of which were also enhanced by the booster vaccination (FIG. 17F, 17G).

Vaccine-induced serum neutralizing activities were evaluated. In the hamster study described earlier, serum samples were collected after booster vaccination (week 5) and prior to viral challenge. Their neutralizing activities against wild-type SARS-CoV-2 (WA1/2020) and the Delta variant were measured by PRNT (Liu et al., Nature 596, 273-75, 2021). Serum from the mRNA-S-vaccinated hamsters manifested strong neutralizing activity against the wild-type virus [wild-type half maximal PRNT values (PRNT₅₀): 2667] but markedly reduced neutralizing activity against the Delta variant (Delta PRNT₅₀: 440; a 5.1-fold reduction) (FIG. 17H). Compared to mRNA-S alone, combination mRNA-S+N elicited stronger serum neutralizing activity against both the wild-type virus (p<0.001) and the Delta variant (p<0.0001) (FIG. 17H). These data are consistent with the augmented S-specific CD8 T cell response (FIG. 17B) induced by combination mRNA-S+N compared to mRNA-S alone. Together, the immune analyses suggest that combination mRNA-S+N vaccination not only induces N-specific immunity, but also elicits stronger S-specific CD8 T cell response and serum neutralizing antibody activities when compared to mRNA-S alone. These responses may collectively contribute to the enhanced protection against the SARS-CoV-2 Delta and Omicron variants.

### B. Material and Methods

mRNA synthesis and LNP formulation. Antigens encoded by the mRNA vaccines were derived from the ancestral SARS-CoV-2 Wuhan-Hu-1 strain (GenBank MN908947.3). Nucleoside-modified mRNAs expressing SARS-CoV-2 full-length N (mRNA-N) or pre-fusion stabilized S protein with two proline mutations (mRNA-S-2P) were synthesized by in vitro transcription using T7 RNA polymerase (MegaScript, Thermo Fisher Scientific) on linearized plasmid templates as previously reported (Pardi et al., Methods Mol Biol 969, 29-42, 2013). UTP was replaced with One-methylpseudouridine (m1′)-5′-triphosphate (TriLink, Cat #N-1081) for producing nucleoside-modified mRNAs. Poly-A tails were added to the end of modified mRNAs for optimized protein expression. In vitro transcribed mRNAs were capped using ScriptCap m7G capping system and ScriptCap 2′-O-methyl-transferase kit (ScriptCap, CellScript) (Pardi et al., Methods Mol Biol 969, 29-42, 2013), followed by purification using the cellulose purification method as previously described (Baiersdorfer et al., Mol Ther Nucleic Acids 15, 26-35, 2019). Purified mRNAs were analyzed by agarose gel electrophoresis and were kept frozen at −20° C. The mRNAs were formulated into lipid nanoparticles (LNP) using an ethanolic lipid mixture of ionizable cationic lipid and an aqueous buffer system as previously reported (Maier et al., Mol Ther 21, 1570-78, 2013; Jayaraman et al., Angew Chem Int Ed Engl 51, 8529-33, 2012). Formulated mRNA-LNPs were prepared according to RNA concentrations (1 μg/μL) and were stored at −80° C. for animal immunizations.

Western blot analysis of protein expression by mRNA-N. 293T cells (American Type Culture Collection, ATCC; CRL-3216) in 6-well plates were directly transfected with 2 μg of mRNA-N-LNP or not transfected (as a cell-only control). Eighteen hours after transfection, cells were lysed in RIPA buffer (Thermo Fisher Scientific) for western blot analysis. Cell lysates were centrifuged, followed by collection of supernatants for quantification of total protein concentration using Microplate bicinchoninic acid (BCA) Protein Assay Kit (Pierce, Thermo Fisher Scientific). Equal amounts of protein were separated by SDS-PAGE using 4-15% SDS polyacrylamide gels (Bio-Rad). Proteins were transferred onto a nitrocellulose membrane (Bio-Rad). The membrane was blocked in tris buffered saline (TBS) containing 0.05% Tween-20 (Thermo Fisher Scientific) (TBST) and 5% (w/v) non-fat dried milk (Bio-Rad) for 1 hour at room temperature, followed by incubation with anti-SARS-CoV2 nucleocapsid mouse monoclonal antibody (MA5-29981, Thermo Fisher Scientific; 1:1000) overnight at 4° C. After washing in TBST (3 times for 5 minutes), the membrane was incubated for 1 hour with horseradish peroxidase (HRP)-linked anti-mouse IgG (7076S, Cell Signaling; 1:5000). The membrane was washed, and proteins were visualized using the ECL Western Blotting Substrate (Thermo Fisher Scientific).

Mouse immunization and SARS-CoV-2 challenge. Vaccine immunogenicity and efficacy were evaluated in 6-week-old female BALB/c mice (Jackson Laboratories; Strain #: 000651). For immunogenicity, four groups of mice (7 per group) were immunized intramuscularly (i.m.) with either PBS (mock control), mRNA-S (1 μg), mRNA-N (1 μg), or combined mRNA-S+N (1 μg for each) at week 0 (prime) and week 3 (boost), respectively. The vaccine or control PBS was administered at 50 μL per injection. Blood and serum samples were collected three weeks after prime vaccination (prior to booster vaccination) to measure vaccine-induced antibody response. All mice were euthanized two weeks after booster vaccination (week 5). Blood and serum and spleen samples were collected for analyses of vaccine-induced humoral and cellular immune responses.

For challenge studies, another four groups of BALB/c mice (8 per group) received the same mock control or vaccines as indicated above. Vaccine doses and immunization timeline were identical to the above immunogenicity study. Two weeks after booster vaccination (week 5), all mice were transferred to animal biosafety level (ABSL)-3 facility and were intranasally challenged with a mouse-adapted SARS-CoV2 CMA4 strain (2×10⁴ pfu) as previously reported (Ku et al., Nat Commun 12, 469, 2021; Muruato et al., PLOS Biol 19, e3001284, 2021) (32, 58). Two days after viral challenge, all mice were euthanized and equivalent portions of the lung tissues were collected for quantification of SARS-CoV-2 viral loads.

Hamster immunization and SARS-CoV-2 Delta or Omicron challenge. Vaccine-induced protection against Delta or Omicron strain was evaluated in hamsters. For Delta challenge, four groups of 4- to 5-week-old male golden Syrian hamsters (12 per group), strain HsdHan: AURA (Envigo; Cat #: 8901M), were vaccinated intramuscularly with either PBS (mock control), mRNA-S (2 μg), mRNA-N (2 μg), or combined mRNA-S+N (2 μg for each) at week 0 and week 3, respectively. For Omicron variant challenge, five groups of 4- to 5-week-old male golden Syrian hamsters (10 per group) were i.m. vaccinated with empty LNP (mock control) mRNA-S (2 μg), mRNA-S (4 μg), mRNA-S+N (2 μg for each), or mRNA-S+N (2 μg for each) with CD8 T cell depletion. For the mRNA-S+N CD8 T cell depleted group, 6 days (Day-6) and 3 days (Day-3) prior to viral challenge, hamsters were intraperitoneally (i.p.) injected with 175 μg anti-rat CD8β antibody (16-0080-38; eBio341; functional grade; Thermo Fisher Scientific) for in vivo CD8 T cell depletion as reported previously (Hammerbeck and Hooper, J Virol 85, 9929-44, 2011; Prescott et al., Immunology 140, 168-78, 2013). CD8 T cell depletion in hamster was confirmed by splenocytes immune staining [anti-CD8β-phycoerythrin (PE); 12-0080-82; eBio341; Thermo Fisher Scientific] and flow cytometric analysis. The vaccine or mock control was administered at 100 μl per injection. Serum samples were collected from all hamsters prior to viral challenge to measure vaccine-induced neutralizing antibodies. Two weeks after booster vaccination (week 5), hamsters were transferred to the ABSL-3 facility and intranasally challenged with the SARS-CoV2 Delta (2×10⁴ pfu) or Omicron strain (2×10⁴ pfu) (World Reference Center for Emerging Viruses and Arboviruses: WRCEVA). Two days post-infection (2 DPI), 6 hamsters challenged with Delta or 5 challenged with Omicron were euthanized. Nasal wash samples and equivalent portions of the lung tissues were collected for various analyses of vaccine-induced protection. On 4 DPI, the same procedures were repeated for the half of hamsters in each group (6 for Delta and 5 for Omicron). Hamster body weights were monitored daily to evaluate vaccine-induced protection from body weight loss.

Binding IgG by ELISA. Vaccine-induced, N- and S-specific binding IgG in serum samples was measured by ELISA. Plates (Greiner bio-one) were coated with 1 μg/mL recombinant S (40589-V08B1; Sino Biological) or N protein (40588-V08B; Sino Biological) overnight at 4° C. Plates were washed three times (5 min each time) and then blocked with blocking buffer [8% fetal bovine serum (FBS) in Dulbecco's phosphate-buffered saline (DPBS)] for 1.5 hour at 37° C., followed by washing and incubation at 37° C. for 1 hour with serially diluted serum samples (initial dilution 1:100; 1:3 serial dilution) in blocking buffer at 50 μL per well. Plates were washed again and incubated with HRP-conjugated anti-mouse IgG secondary antibody (405306; BioLegend; 1:3000) for 1 hour at 37° C. After final wash, plates were developed using TMB 1-Component Peroxidase Substrate (Thermo Fisher Scientific), followed by termination of reaction using the TMB stop solution (Thermo Fisher Scientific). Plates were read at 450 nm wavelength within 15 minutes by using a Microplate Reader (BioTek). Binding IgG Endpoint titers (EPT) for each sample were calculated.

Neutralizing assay. Serum neutralizing activity was examined by a standard Plaque Reduction Neutralization Test (PRNT) as previously reported (Xic et al., Nat Med, 2021; Muruato et al., Nat Commun 11, 4059, 2020). The assays were performed with Vero E6 cells (ATCC; CRL-1586) using the SARS-CoV-2 wild-type or Delta strains. In brief, serum samples were heat-inactivated, and two-fold serially diluted (initial dilution 1:10), followed by incubation with 100 pfu wild-type SARS-CoV2 (USA-WA1/2020) or the Delta strain for 1 hour at 37° C. The serum-virus mixtures were placed onto Vero E6 cell monolayer in 6-well plates for incubation for 1 hour at 37° C., followed by addition of 2 ml overlay consisting of MEM with 1.6% agarose, 2% FBS and 1% penicillin-streptomycin to the cell monolayer. Cells were then incubated for 48 hours at 37° C., followed by staining with 0.03% liquid neutral red for 3 to 6 hours. Plaque numbers were counted and PRNT₅₀ were calculated. Each serum sample was tested in duplicates.

Intracellular Cytokine Staining (ICS) and Flow Cytometry. Mouse splenocytes were washed with FACS buffer (1% FBS and 0.5 M EDTA in PBS) and resuspended in complete RPMI-1640 with 10 mM HEPES supplemented with 10% FBS, 2-Mercaptoethanol, Sodium Pyruvate, Non-Essential Amino Acids, Pen-Strep, and L-Glutamine. Cells were stimulated with 1 μg/mL S peptide pool (JPT, PM-WCPV-S) (Swiss-Prot ID: PODTC2) or N peptide pool (Miltenyi, 130-126-698) (Protein QHD43423.2) in the presence of 1 g/mL anti-CD28 (Invitrogen, 14-0281-86) for co-stimulation for 6 hours. In the last 4 hours of incubation, protein transport inhibitor Brefeldin-A was added. Cells stimulated with phorbol 12-myristate 13-acetate (PMA) and ionomycin or dimethyl sulfoxide only were included as positive control and negative control, respectively. Following stimulation, cells were first stained for surface markers, including CD4-peridinin-chlorophyll-protein (PerCP)-Cy5.5 (BioLegend, 100540; Clone: RM4-5; 0.2 mg/ml), CD8-brillaint violet (BV) 711 (BioLegend, 100759; Clone: 53-6.7; 0.2 mg/ml), and CD44-BV510 (BioLegend, 103044; Clone: IM7; 0.2 mg/ml). The surface staining was performed on ice for 30 minutes. After washing with PBS, cells were resuspended with Zombie-dye (BioLegend) for viability staining and incubated at room temperature for 15 minutes. Following surface and viability staining, cells were fixed with fixation buffer (BioLegend, 420801) and permeabilized with perm/wash buffer (BioLegend, 421002), followed by intracellular cytokine staining with IFN—γ-BV605 (BioLegend, 505840; Clone: XMG1.2; 0.2 mg/ml), TNF-α-PE-Cy7 (BioLegend, 506324; Clone: MP6-XT22; 0.2 mg/ml), and IL-2-allophycocyanin (APC) (Tonbo bioscience, 20-7021; Clone: JES6-5H4; 0.2 mg/ml) on ice for 30 minutes. Cells were then washed with perm/wash buffer and were processed with a multi-parametric flow cytometer FACS LSR Fortessa (BD Biosciences). Data were analyzed using FlowJo (TreeStar).

IFN-γ ELISPOT. ELISPOT was performed according to manufacturer's instructions (Cellular Technology Ltd; MU IFN-γ). Plates were coated with anti-IFN-γ capture antibody (Cellular Technology Ltd) at 4° C. overnight. Splenocytes (0.25×10⁶) were stimulated in duplicates with SARS-CoV-2 S-(2 μg/mL, Miltenyi Biotec, 130-126-701) or N-peptide pools (2 μg/mL, Miltenyi Biotec, 130-126-699) for 24 hours at 37° C. Splenocytes stimulated with anti-CD3 (1 μg/mL, Thermo Fisher Scientific, 16-0031-82) or medium alone were used as positive and negative control, respectively. This was followed by incubation with biotin-conjugated anti-IFN-γ (Cellular Technology Ltd) for 2 hours at room temperature, and then alkaline phosphatase-conjugated streptavidin for 30 minutes. The plates were washed and scanned using an ImmunoSpot 4.0 analyzer and the spots were counted with ImmunoSpot software (Cellular Technology Ltd) to determine SFC per 10⁶ splenocytes.

RNA extraction and qPCR quantification of viral loads. RNA was extracted from the lung tissues (mice and hamsters) and nasal washes (hamsters) using the TRIzol LS reagent (Thermo Fisher Scientific) according to the manufacturer's instructions. Concentration and purity of the extracted RNAs were determined using the multi-mode plate reader (BioTek). To quantify SARS-CoV-2 viral RNA copies, one-step RT-PCR was performed using the iTaq Universal SYBR Green One-Step Kit (Bio-Rad) and the CFX Connect Real-Time PCR Detection System (Bio-Rad). Primer sets for the SARS-CoV-2 E gene (F: 5′-GGAAGAGACAGGTACGTTAATA-3′ (SEQ ID NO: 7); R: 5′-AGCAGTACGCACACAATCGAA-3′ (SEQ ID NO:8)) were used. PCR reactions (20 μL) contained primers (10 μM), RNA sample (2 μL), iTaq universal SYBR Green reaction mix (2×) (10 μL), iScript reverse transcriptase (0.25 μL), and molecular grade water. PCR cycling conditions were: 95° C. for 3 minutes, 45 cycles of 95° C. for 5 seconds, and 60° C. for 30 seconds. For each RT-PCR, a standard curve was included, using an RNA standard (in vitro transcribed, 3,839 bp containing genomic nucleotide positions 26,044 to 29,883 of SARS-CoV-2 genome), to quantify the absolute copies of viral RNA in the lung tissue or nasal wash.

Plaque assay. Homogenized lung tissues were serially diluted in Dulbecco's Modified Eagle Medium (DMEM, Gibco) with 1% antibiotic-antimycotic (Gibco) and allowed to infect a confluent monolayer of Vero E6 cells (ATCC; CRL-1586) in a 96-well plate for 45 minutes at 37° C. with 5% CO₂. Following infection, cells were overlaid with a solution of 85% Minimum Essential Media (MEM, Gibco) and 15% DMEM supplemented with 1% antibiotic-antimycotic and 0.85% methyl cellulose (Sigma-Aldrich). After 24 to 36 hours, the monolayers were fixed with formalin (Thermo Fisher Scientific) for at least 24 hours. Monolayers were washed with DPBS (Sigma) and incubated in permeabilization buffer consisting of DPBS supplemented with 0.1% bovine serum albumin (Sigma-Aldrich) and 0.1% saponin (Sigma-Aldrich) for 30 minutes at room temperature. Permeabilization buffer was removed, and monolayers were incubated overnight at 4° C. with rabbit polyclonal antibody against SARS-CoV N protein (A gift from Shinji Makino, Department of Microbiology & Immunology, UTMB) diluted in permeabilization buffer (1:3000). Excess antibody was washed away with DPBS, and monolayers were incubated for one hour at room temperature with HRP-conjugated goat anti-rabbit IgG (Cell Signaling, 7040) diluted in permeabilization buffer (1:2000). Excess antibody was washed away with DPBS, and foci were stained using KPL TrueBlue Peroxidase Substrate (SeraCare). Once foci were visible under a light microscope, excess substrate was removed and the monolayers were washed with water. Wells were imaged using the Cytation7 Imagining Reader (BioTek). Foci were counted manually and results were shown as focus-forming units (FFU).

Lung histopathology. Lungs were harvested from hamsters, fixed in 10% neutral-buffered formalin, and embedded in paraffin. Thin (5 μm) paraffin-embedded sections were placed on glass slides, and paraffin was then removed from the samples using three changes of xylene for two minutes each. Samples were hydrated, followed by staining for 3 minutes in hematoxylin solution. The slides were then washed under running tap water at room temperature for at least 5 minutes, followed by staining with an eosin Y solution for 2 minutes. Slides were then subjected to dehydration again and cleared with three changes of xylene for 2 minutes per change. Finally, a drop of mounting medium was added to attach the coverslip. The slides were read by a pathologist in a blinded manner.

Statistical Analysis. Statistical analyses were performed using Graph-Pad Prism 8.0. Statistical comparison was performed using either unpaired Student's t test or one-way ANOVA where appropriate. The values were presented either as mean or mean±SD. Two-tailed p values were denoted, and p values <0.05 were considered significant.

1. A SARS-CoV-2 vaccine, comprising an engineered messenger ribonucleic acid (mRNA) comprising an open reading frame encoding a coronavirus nucleoprotein (N) protein.

2. A SARS-CoV-2 vaccine, comprising an engineered messenger ribonucleic acid (mRNA) comprising an open reading frame encoding a coronavirus spike(S) protein.

3. The vaccine of claim 1, further comprising an engineered messenger ribonucleic acid (mRNA) comprising an open reading frame encoding a coronavirus spike(S) protein.

4. The vaccine of claim 1, wherein the N protein is encoded by a nucleic acid segment that is 90, 95, 99, or 100% identical to SEQ ID NO:3.

5. The vaccine of claim 1, wherein the S protein is encoded by a nucleic acid segment that is 90, 95, 99, or 100% identical to SEQ ID NO: 6.

6. The vaccine of claim 1, wherein the mRNA is linear.

7. The vaccine of claim 1, further comprising a 5′ UTR.

8. The vaccine of claim 1, further comprising a 3′ UTR.

9. The vaccine of claim 1, further comprising a polyadenylation segment.

10. A DNA construct encoding the mRNA of claim 1.

11. A vaccine composition comprising an mRNA of claim 1 comprised in a lipid nanoparticle (LNP).

12. The composition of claim 11, wherein the LNP comprising an ionizable cationic lipid, phosphatidylcholine, cholesterol, and PEG-lipid.

13. The composition of claim 11, comprising the vaccine of any one of claim 1.

14. The composition of claim 11, comprising the vaccine of claim 1, claim 2, or claim 3.

15. A method of inducing an antigen-specific immune response in a subject, the method comprising administering to the subject the vaccine of claim 1 to produce an antigen-specific immune response in the subject.

16. The method of claim 15 wherein the vaccine is administered using a prime-boost regimen.

17. The method of claim 16, wherein the boost dose is administered 2, 3, 4, 5 or 6 weeks after the prime dose.
